(19) United States
(12) Patent Application Publication
(10) Pub. No.: US 2024/0009173 A1
(43) Pub. Date: Jan. 11, 2024

(54) Use of 5-HT3-Targeting Drugs for Treatment of Acute Kidney Injury

(71) Applicant: University of Pittsburgh-Of the Commonwealth System of Higher Education, Pittsburgh, PA (US)

(72) Inventors: LiRong Wang, McDonald, PA (US); Xiguang Qi, Pittsburgh, PA (US); Xiaojiang Guo, Pittsburgh, PA (US); Peihao Fan, Pittsburgh, PA (US); John A. Kellum, Pittsburgh, PA (US)

(21) Appl. No.: 18/035,152

(22) PCT Filed: Nov. 5, 2021
(86) PCT No.: PCT/US21/58278

§ 371 Date: May 3, 2023

Related U.S. Application Data
(60) Provisional application No. 63/110,072, filed on Nov. 5, 2020.

(51) Int. Cl. A61K 31/4178; A61P 13/12; A61K 45/06

(52) U.S. Cl. CPC A61K 31/4178; A61P 13/12; A61K 45/06

(57) ABSTRACT

A method of treating acute kidney injury in a patient is provided, including administering to the patient a 5-HT₃-targeting drug, the 5-HT_{3A}-targeting drug, or the pharmaceutically-acceptable salt or solvate of a 5-HT₃-targeting drug or a 5-HT_{3A}-targeting drug, in an amount effective to treat acute kidney injury in the patient.

### CROSS-REFERENCE TO RELATED APPLICATIONS

This application is the United States national phase of International Application No. PCT/US21/58278 filed Nov. 5, 2021, and claims priority to U.S. Provisional Application No. 63/110,072, filed Nov. 5, 2020, the disclosures of which are hereby incorporated by in their entireties.

### BACKGROUND OF THE INVENTION

Acute kidney injury (AKI), also called acute renal failure or acute kidney failure, is a common renal disorder worldwide and is encountered in various clinical settings. In the United States, more than 60% of patients will suffer from AKI during the intensive care unit (ICU) admission. AKI also give rise to an extensive mortality to 20-50% in critically ill patients in ICU and 1 year afterwards. AKI is generally diagnosed as an unexpected and usually reversible decline in glomerular filtration, which is mostly associated with anemia and reperfusion of kidney with various causes. Patients with AKI are at high risk of poor prognosis such as prolonged ICU and hospitalization stays, significantly elevated risk of mortality within 1 year after ICU admission, future development of chronic kidney disease (CKD), and possible need of kidney transplant surgery. Given the high mortality and poor prognosis of AKI in ICU patients, there is an extensive amount of previous research to identify effective treatments for AKI. Unfortunately, the pathology of AKI is still not very clear and no drug pertaining to the pathology of AKI has been found in clinical practice.

Therapy of critically ill patients with AKI demands collaboration in numbers of treatment across multiplex disciplines. In 2013, Kidney Disease: Improving Global Outcomes (KDIGO) published the first interdisciplinary and international clinical practice guideline on AKI (Kellum, J. A. et al. “Kidney disease: improving global outcomes (KDIGO) acute kidney injury work group. KDIGO clinical practice guideline for acute kidney injury”, Kidney International Supplements 2012, 2:1-138). As illustrated by the guideline, recommendations were given on administrations in the practice of critical care. Several treatments were recommended by the guideline, including insulin therapy targeting plasma glucose 110 to 149 mg/dl in critically ill AKI patients, implementation of a total energy intake of 20 to 30 kcal/kg/day in any AKI patients (nutrition is recommended through the enteral route), preventing reduction of protein intake of patients to delay or avoid renal replacement therapy, and antifungal and/or echinocandin therapy, rather than amphotericin B, in the treatment of systemic mycoses or parasitic infections. On the other hand, several currently widely used treatments such as using diuretics (except for the management when volume is overload), low-dose dopamine, fenoldopam, ANP (atrial natriuretic peptide), and recombinant human insulin-like growth factor-1 were not suggested by the guideline to prevent or treat AKI. Aminoglycosides were not suggested to be used for the treatment of infections unless no suitable, less nephrotoxic therapeutic alternatives are available.

Treatments for AKI and critically ill patients at risk of developing AKI are needed.

### SUMMARY

A method for treatment of acute kidney injury in a patient in need thereof is provided. The method comprises administering to the patient a 5-HT₃-targeting drug, a 5-HT_{3A}-targeting drug, or a pharmaceutically-acceptable salt or solvate of a 5-HT₃-targeting drug or a 5-HT_{3A}-targeting drug, in an amount effective to treat acute kidney injury in the patient.

A method for treatment of a critically ill patient (a patient having one or more life-threatening conditions, e.g., requiring critical care service or intensive care service) also is provided. The method comprises administering to the patient a 5-HT₃-targeting drug, a 5-HT_{3A}-targeting drug, or a pharmaceutically-acceptable salt or solvate of a 5-HT₃-targeting drug or a 5-HT_{3A}-targeting drug, in an amount effective to reduce risk of acute kidney injury in the patient.

The following numbered clauses describe various aspects and/or embodiments of the present invention.
Clause 1. A method for treatment of acute kidney injury in a patient in need thereof, comprising administering to the patient a 5-HT₃-targeting drug, a 5-HT_{3A}-targeting drug, or a pharmaceutically-acceptable salt or solvate of a 5-HT₃-targeting drug or a 5-HT_{3A}-targeting drug, in an amount effective to treat acute kidney injury in the patient.
Clause 2. A method for treatment of a critically ill patient (a patient having one or more life-threatening conditions, e.g., requiring critical care service or intensive care service), comprising administering to the patient a 5-HT₃-targeting drug, a 5-HT_{3A}-targeting drug, or a pharmaceutically-acceptable salt or solvate of a 5-HT₃-targeting drug or a 5-HT_{3A}-targeting drug, in an amount effective to reduce risk of acute kidney injury in the patient.
Clause 3. The method of clause 2, wherein the critically ill patient is: an intensive care unit patient, a critical care unit patient, a patient requiring intensive care or critical care, a sepsis patient, a patient recovering from major surgery (a surgery in which a body cavity is entered), a patient exposed to a nephrotoxin, a patient having a microbial infection that increases risk of acute kidney injury, a kidney transplant recipient, a burn patient, or a critically-injured patient.
Clause 4: The method of any one of clauses 1 to 3, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is a 5-HT₃ antagonist, a 5-HT_{3A} antagonist, or a pharmaceutically-acceptable salt or solvate of a 5-HT₃ antagonist or a 5-HT_{3A} antagonist.
Clause 5. The method of any one of clauses 1 to 3, wherein 5-HT₃-targeting drug, the 5-HT_{3A}-targeting drug, or the pharmaceutically-acceptable salt or solvate of a 5-HT₃-targeting drug or a 5-HT_{3A}-targeting drug is one or more of tubocurarine, clozapine, aripiprazole, olanzapine, and metoclopramide, or a pharmaceutically-acceptable salt or solvate of any of the preceding.
Clause 6. The method of any one of clauses 1 to 3, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is one or more of ondansetron, granisetron, dolasetron, palonosetron, alosetron, cilansetron, tropisetron, ramosetron, or a pharmaceutically-acceptable salt or solvate of any of the preceding.
Clause 7. The method of any one of clauses 1 to 3, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is ondansetron, or a pharmaceutically-acceptable salt or solvate thereof.
Clause 8. The method of any one of clauses 1 to 7, wherein the patient is in an intensive care unit.
Clause 9. The method of any one of clauses 1 to 8, wherein the patient has a viral infection.
Clause 10. The method of any one of clauses 1 to 10, wherein the patient has a coronavirus infection.
Clause 11. The method of any one of clauses 1 to 10, wherein the patient has a SARS-CoV-2 coronavirus infection (COVID-19).
Clause 12. The method of any one of clauses 1 to 11, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug reduces mortality or increases survival in a population of patients having acute kidney injury.
Clause 13. The method of any one of clauses 1 to 12, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is administered once acute kidney injury is detected in the patient, when serum creatinine of the patient increases by 0.3 mg/dl (26.5 μmol/1) or more in 48 hours, when serum creatinine of the patient rises to at least 1.5-fold from baseline within 7 days; when urine output is less than 0.5 ml/kg/hr for more than 6 hours (e.g., KDIGO criteria), or when any other approved diagnostic for AKI is positive.
Clause 14. The method of any one of clauses 1 to 13, wherein the 5-HT₃-targeting drug, 5-HT_{3A}-targeting drug, or a pharmaceutically-acceptable salt or solvate thereof is administered to the patient until serum creatinine levels are normalized in the patient.
Clause 15. The method of any one of clauses 1 to 13, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is administered by intravenous injection or infusion.
Clause 16. The method of any one of clauses 1 to 15, wherein a total of from 0.5 mg to 200 mg of the 5-HT₃-targeting drug, the 5-HT_{3A}-targeting drug, or the pharmaceutically-acceptable salt or solvate of a 5-HT₃-targeting drug or a 5-HT_{3A}-targeting drug is administered daily to the patient, such as in one or more doses or as a continuous or intermittent infusion.
Clause 17. The method of clause 16, wherein a total of from 6 mg to 96 mg of ondansetron, or a pharmaceutically-acceptable salt or solvate thereof is administered daily to the patient.
Clause 18. The method of clause 17, wherein from 4 mg to 32 mg of ondansetron, or a pharmaceutically-acceptable salt or solvate thereof is administered every 8 hours to the patient.
Clause 19. The method of any one of clauses 1 to 18, further comprising administering to the patient one or more drugs selected from the group consisting of: warfarin; Lisinopril; heparin; magnesium sulfate; haloperidol; glucagon; metoprolol; furosemide; and hydralazine, or a pharmaceutically-acceptable salt or solvate of any of the preceding.

### BRIEF DESCRIPTION OF THE DRAWINGS

FIG. 1 is a flow chart of procedure described in Example 1.

FIG. 2 depicts the acute physiology scores (APS) distribution of patients that took (treated units)/did not take (control units) ondansetron before and after PSM.

FIG. 3 depicts the distribution of matched/unmatched samples in PSM process according to propensity scores.

FIG. 4 provides a detailed comparison between ondansetron-induced gene expression profile and kidney from transplant patient with toxic drug effects.

FIGS. 5A-5C provide graphs showing serum creatinine levels following administration of Ondansetron, Magnesium Sulfate and Aspirin. In these three graphs, the X axis is time in hours for calculating the slopes of creatinine changes after the drug (Ondansetron, Magnesium Sulfate and Aspirin) use, and Y axis is the slope.

FIGS. 6A-6C are flowcharts of the procedures. FIG. 6A is a flowchart showing the overview of the experimental design. FIG. 6B is a flowchart of AKI patient inclusion in the MIMIC III database. FIG. 6C is a flowchart of AKI patient inclusion in the eICU database.

FIGS. 7A-7C are plots showing the changes of HT₃ receptor genes HTR3A (FIG. 7A), HTR3B (FIG. 7B), and HTR3C (FIG. 7C) in patients with AKI compared with control (the pristine protocol biopsies).

FIG. 8 is a volcano plot of gene expression changes of AKI compared with the control.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include but are not limited to embodiments “consisting essentially of” and “consisting of” these stated elements or steps.

A method of treating a patient with acute kidney injury is provided. The method comprises administering to a patient having acute kidney injury a 5-HT₃-targeting drug, a 5-HT_{3A}-targeting drug, or a pharmaceutically-acceptable salt or solvate thereof, in an amount effective to treat acute kidney injury in a patient, or in the patient. The acute kidney injury may be associated with any injury or disease, including traumatic injury, exposure to a toxin or radiation, post-surgical trauma, cancer, a bacterial, viral or fungal infection. The patient may have a viral infection, such as a coronavirus infection, such as including MERS-CoV or SARS-CoV-2 (COVID-19). The 5-HT₃-targeting drug, 5-HT_{3A}-targeting drug, or pharmaceutically-acceptable salt or solvate thereof may be ondansetron, or a pharmaceutically-acceptable salt or solvate thereof.

A 5-HT₃ targeting drug includes agonists, antagonists, or specific ligands of the 5-HT₃ receptor (e.g., serotonin receptor 3A), such as 5-HT₃-targeting drugs or 5-HT_{3A}-targeting drugs. 5-HT₃-targeting drugs or 5-HT_{3A}-targeting drugs include 5-HT₃ antagonists and 5-HT_{3A} antagonists, which are a broadly-known class of drugs (e.g., active pharmaceutical ingredient), some of which have found use as an antiemetic, e.g., in the treatment of nausea and vomiting, and generally have been approved for their antiemetic activity for example for treatment of chemotherapy-induced nausea/vomiting (CINV), radiation-induced emesis (RIS), and postoperative nausea/vomiting (PONV). 5-HT₃ antagonists and/or 5-HT_{3A} antagonists include ondansetron, granisetron, dolasetron, palonosetron, alosetron, cilansetron, tropisetron, and ramosetron (see, e.g., Smith et al. “5-HT₃ receptor antagonists for the treatment of nausea/vomiting”, Ann Palliat Med., 2012, 1(2):115-20, see also Thompson et al. “5-HT₃ receptors,” Current Pharmaceutical Design, 2006, 12(28): 3615-3630, describing the 5-HT₃ receptor). 5-HT₃-targeting drugs or 5-HT_{3A}-targeting drugs, such as 5-HT₃ antagonists and 5-HT_{3A} antagonists include any free base or pharmaceutically-acceptable salt or solvate thereof, for example ondansetron includes ondansetron, ondansetron hydrochloride, and other pharmaceutically acceptable salts of ondansetron. Other exemplary 5-HT₃-targeting drugs or 5-HT_{3A}-targeting drugs include: tubocurarine, clozapine, aripiprazole, olanzapine, and metoclopramide.

Use of 5-HT₃ targeting drugs are described herein for the treatment of patients with acute kidney injury, such as trauma patients or ICU patients. The drugs are administered in an amount effective to reduce AKI-associated mortality, and/or to improve at least one aspect of kidney function in an AKI-diagnosed patient.

As used herein, a “patient” is an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose).

As used herein, the terms “treating”, or “treatment” refer to a beneficial or desired result, such as improving one of more, or symptoms of a disease. The terms “treating” or “treatment” also include, but are not limited to, alleviation or amelioration of one or more symptoms of acute kidney injury. “Treatment” can also mean prolonging survival as compared to expected survival in the absence of treatment.

By “lower” in the context of a disease marker or symptom is meant a clinically-relevant and/or a statistically significant decrease in such level. The decrease can be, for example, at least 10%, at least 20%, at least 30%, at least 40%, or more, down to a level accepted as within the range of normal for an individual without such disorder, or to below the level of detection of the assay. In certain aspects, the decrease is down to a level accepted as within the range of normal for an individual without such disorder which can also be referred to as a normalization of a level. In certain aspects, the reduction is the normalization of the level of a sign or symptom of a disease, a reduction in the difference between the subject level of a sign of the disease and the normal level of the sign for the disease (e.g., to the upper level of normal when the value for the subject must be decreased to reach a normal value, and to the lower level of normal when the value for the subject must be increased to reach a normal level).

“Therapeutically effective amount,” or an “amount effective” as used herein, is intended to include the amount of a drug as described herein that, when administered to a subject having a disease, is sufficient to effect treatment of the disease (e.g., by diminishing, ameliorating or maintaining the existing disease or one or more symptoms of disease). The “therapeutically effective amount” may vary depending on the nature of the injury and its causes, how the agent is administered, the disease and its severity and the history, age, weight, family history, genetic makeup, the types of preceding or concomitant treatments, if any, and other individual characteristics of the subject to be treated. In the context of the drugs described herein, an exemplary dosage range, per day, ranges from 0.5 μg (micrograms) to 200 mg (milligrams), for example 500 μg to 150 mg per day. A “therapeutically-effective amount” also includes an amount of a drug that produces some desired local or systemic effect at a reasonable benefit/risk ratio applicable to any treatment.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates, and/or polyanhydrides; (22) bulking agents, such as polypeptides and amino acids (23) serum component, such as serum albumin, HDL and LDL; and (22) other non-toxic compatible substances employed in pharmaceutical formulations.

Pharmaceutically acceptable salts of any of the compounds described herein also may be used in the methods described herein. Pharmaceutically acceptable salt forms of the compounds described herein may be prepared by conventional methods known in the pharmaceutical arts, and include as a class veterinarily acceptable salts. For example and without limitation, where a compound comprises a carboxylic acid group, a suitable salt thereof may be formed by reacting the compound with an appropriate base to provide the corresponding base addition salt. Non-limiting examples include: alkali metal hydroxides, such as potassium hydroxide, sodium hydroxide and lithium hydroxide; alkaline earth metal hydroxides, such as barium hydroxide and calcium hydroxide; alkali metal alkoxides, such as potassium ethanolate and sodium propanolate; and various organic bases such as piperidine, diethanolamine, and N-methylglutamine.

Non-limiting examples of pharmaceutically-acceptable base salts include: aluminum, ammonium, calcium, copper, ferric, ferrous, lithium, magnesium, manganic, manganous, potassium, sodium, and zinc salts. Salts derived from pharmaceutically acceptable organic non-toxic bases include, without limitation: salts of primary, secondary, and tertiary amines, substituted amines including naturally occurring substituted amines, cyclic amines, and basic ion exchange resins, such as arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

Non-limiting examples of pharmaceutically-acceptable acid salts include: acetate, adipate, alginate, arginate, aspartate, benzoate, besylate (benzenesulfonate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, and phthalate.

Multiple salts forms are also considered to be pharmaceutically-acceptable salts. Common, non-limiting examples of multiple salt forms include: bitartrate, diacetate, difumarate, dimeglumine, diphosphate, disodium, and trihydrochloride.

As such, “pharmaceutically acceptable salt” as used herein is intended to mean an active ingredient (drug) comprising a salt form of any compound as described herein. The salt form preferably confers to the improved and/or desirable pharmacokinetic/pharmodynamic properties of the compounds described herein.

Organic compounds can form complexes with solvents in which they are reacted or from which they are precipitated or crystallized. These complexes are known as “solvates.” A complex with water is known as a “hydrate.” Solvates of the 5-HT₃-targeting drug, 5-HT_{3A}-targeting drug are contemplated. It will also be appreciated by those skilled in organic chemistry that many organic compounds can exist in more than one crystalline form. For example, crystalline form may vary from solvate to solvate. Thus, all crystalline forms of 5-HT₃-targeting drug, 5-HT_{3A}-targeting drug or the pharmaceutically acceptable solvates thereof are within the scope of the present invention (See, generally, A. M. Healy, et al., “Pharmaceutical solvates, hydrates and amorphous forms: A special emphasis on cocrystals”, Adv. Drug Deliv. Rev. 2017, 117:25-46).

The compositions described herein can be administered by any effective route. Examples of delivery routes include, without limitation: topical, for example, epicutaneous, inhalational, enema, ocular, otic, and intranasal delivery; enteral, for example, orally, by gastric feeding tube, and rectally; and parenteral, such as, intravenous, intraarterial, intramuscular, intracardiac, subcutaneous, intraosseous, intradermal, intrathecal, intraperitoneal, transdermal, iontophoretic, transmucosal, epidural, and intravitreal, with oral, intravenous, intramuscular, and transdermal approaches being preferred in many instances. Suitable dosage forms may include single-dose, or multiple-dose vials or other containers, such as medical syringes, containing a composition comprising an active ingredient useful for treatment of acute kidney injury, as described herein.

Dosage regimens may be adjusted to provide the optimum desired response (e.g., a therapeutic or prophylactic response). For example, a single bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions, such as parenteral or inhaled compositions, in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Useful dosage forms include: intravenous, intramuscular, or intraperitoneal solutions, oral tablets, capsules, or liquids, topical ointments or creams, and transdermal devices (e.g., patches). The compound may be provided in a sterile solution comprising the active ingredient (drug, or compound), and a solvent, such as water, saline, lactated Ringer's solution, or phosphate-buffered saline (PBS), e.g., as a parenteral dosage form, such as an intravenous infusion or injection. Additional excipients, such as polyethylene glycol, emulsifiers, salts and buffers may be included in the solution.

Therapeutic/pharmaceutical compositions are prepared in accordance with acceptable pharmaceutical procedures, such as described in Remington: The Science and Practice of Pharmacy, 21^{st} edition, ed. Paul Beringer et al., Lippincott, Williams & Wilkins, Baltimore, MD Easton, Pa. (2005) (see, e.g., Chapters 37, 39, 41, 42 and 45 for examples of powder, liquid, parenteral, intravenous and oral solid formulations and methods of making such formulations).

In some embodiments, the 5-HT₃-targeting drug or 5-HT_{3A}-targeting drug is tubocurarine, a pharmaceutically-acceptable salt of tubocurarine, or a solvate of tubocurarine and is available as an intravenous injectable solution, as approved by the Food and Drug Administration (FDA). Injectable solutions include an effective amount of tubocurarine ranging from at least about 0.5 milligrams per milliliters (mg/mL) to at least about 4 mg/mL, from at least about 0.8 mg/mL to at least about 3.5 mg/mL, from at least about 1 mg/mL to at least about 3 mg/mL, or from at least about 1.5 mg/mL to at least about 3 mg/mL. In some cases, administration of tubocurarine, a pharmaceutically-acceptable salt of tubocurarine, or a solvate of tubocurarine is at a dose from at least about 0.05 mg per kilogram per day (mg/kg d) to at least about 0.25 mg/kg d. In some embodiments, administration of tubocurarine, a pharmaceutically-acceptable salt of tubocurarine, or a solvate of tubocurarine is at a dose of at least about 0.2 mg/kg d.

In some embodiments, the 5-HT₃-targeting drug or 5-HT_{3A}-targeting drug is clozapine, a pharmaceutically-acceptable salt of clozapine, or a solvate of clozapine and is available in several dosage forms (e.g., oral tablets or an oral suspension), as approved by the FDA. Oral tablets or orally disintegrating tablets include an effective amount of clozapine ranging from at least about 12.5 milligrams (mg) to at least about 200 mg, from at least about 12.5 mg to at least about 100 mg, from at least about 12.5 mg to at least about 50 mg, or from at least about 12.5 mg to at least about 25 mg. Oral suspensions include an effective amount of clozapine ranging from at least about 25 mg/mL to at least about 50 mg/mL. In some embodiments, the oral suspension includes clozapine in an effective amount of at least about 50 mg/mL. In some cases, administration of clozapine, a pharmaceutically-acceptable salt of clozapine, or a solvate of clozapine is at a dose ranging from at least about 12.5 mg per day to at least 600 mg per day. In some embodiments, administration of clozapine, a pharmaceutically-acceptable salt of clozapine, or a solvate of clozapine is at a dose ranging from at least about 25 mg per day to at least about 300 mg per day.

In some embodiments, the 5-HT₃-targeting drug or 5-HT_{3A}-targeting drug is aripiprazole, a pharmaceutically-acceptable salt of aripiprazole, or a solvate of aripiprazole and is available in several dosage forms (e.g. oral tablets, oral suspensions, or intramuscular injections, which can be used alone or in combination), as approved by the FDA. Oral tablets may include an effective amount of aripiprazole ranging from at least about 2 mg to at least about 30 mg, from at least about 2 mg to at least about 25 mg, from at least about 2 mg to at least about 20 mg, from at least about 2 mg to at least about 15 mg, from at least about 2 mg to at least about 10 mg, from at least about 2 mg to at least about 5 mg, from at least about 5 mg to at least about 30 mg, from at least about 5 mg to at least about 25 mg, from at least about 5 mg to at least about 20 mg, from at least about 5 mg to at least about 15 mg, from at least about 5 mg to at least about 10 mg, from at least about 10 mg to at least about 30 mg, from at least about 10 mg to at least about 25 mg, from at least about 10 mg to at least about 20 mg, from at least about 10 mg to at least about 15 mg, from at least about 15 mg to at least about 30 mg, from at least about 15 mg to at least about 25 mg, from at least about 15 mg to at least about 20 mg, from at least about 20 mg to at least about 30 mg, from at least about 20 mg to at least about 25 mg, from at least about 25 mg to at least about 30 mg. Oral suspensions may include an effective amount of aripiprazole ranging from at least about 0.25 mg/mL to at least about 2 mg/mL, from at least about 0.5 mg/mL to at least about 1.5 mg/mL, from at least about 0.75 mg/mL to at least about 1.25 mg/mL, or at least about 1 mg/mL. Intramuscular injections may include an effective amount of aripiprazole ranging from at least about 0.5 mg/mL to at least about 400 mg/mL, from at least about 1 mg/mL to at least about 300 mg/mL, from at least about 50 mg/mL to at least about 300 mg/mL, from at least about 100 mg/mL to at least about 300 mg/mL, from at least about 200 mg/mL to at least about 300 mg/mL, or from at least about 250 mg/mL to at least about 300 mg/mL. In some cases, administration of aripiprazole, a pharmaceutically-acceptable salt of aripiprazole, or a solvate of aripiprazole is at a dose ranging from at least about 10 mg per day to at least about 30 mg per day, from at least about 15 mg per day to at least about 30 mg per day, or from at least about 10 mg per day to at least about 15 mg per day.

In some embodiments, the 5-HT₃-targeting drug or 5-HT_{3A}-targeting drug is olanzapine, a pharmaceutically-acceptable salt of olanzapine, or a solvate of olanzapine and is available in several dosage forms (e.g., oral tablets or intramuscular injections, which can be used alone or in combination), as approved by the FDA. Oral tablets may include an effective amount of olanzapine ranging from at least about 2.5 mg to at least about 20 mg, from at least about 2.5 mg to at least about 15 mg, from at least about 2.5 mg to at least about 10 mg, from at least about 2.5 mg to about 7.5 mg, from at least about 2 mg to at least about 5 mg, from at least about 5 mg to at least about 20 mg, from at least about 5 mg to at least about 15 mg, from at least about 5 mg to at least about 10 mg, from at least about 5 mg to at least about 7.5 mg, from at least about 7.5 mg to at least about 20 mg, from at least about 7.5 mg to at least about 15 mg, from at least about 7.5 mg to at least about 10 mg, from at least about 10 mg to at least about 20 mg, from at least about 10 mg to at least about 15 mg, or from at least about 15 mg to at least about 20 mg. Intramuscular injections may include an effective amount of olanzapine ranging from at least about 10 mg per vial (mg/vial) to at least about 410 mg/vial, from at least about 50 mg/vial to at least about 400 mg/vial, from at least about 100 mg/vial to at least about 410 mg/vial, from at least 200 mg/vial to at least 400 mg/vial, or from at least 300 mg/vial to at least 400 mg/vial. In some cases, administration of olanzapine, a pharmaceutically-acceptable salt of olanzapine, or a solvate of olanzapine is at a dose ranging from at least about 5 mg per day to at least about 20 mg per day, from at least about 10 mg per day to at least about 20 mg per day, or from at least about 15 mg per day to at least about 20 mg per day.

In some embodiments, the 5-HT₃-targeting drug or 5-HT_{3A}-targeting drug is metoclopramide, a pharmaceutically-acceptable salt of metoclopramide, or a solvate of metoclopramide and is available in several dosage forms (e.g., oral tablets, injectable solution, or oral solution), as approved by the FDA. Oral tablets may include an effective amount of metoclopramide ranging from at least about 5 mg to at least about 10 mg. Oral suspensions may include an effective amount of metoclopramide of at least about 5 mg/mL and injectable solutions may include an effective amount of metoclopramide of at least about 5 mg/mL. In some cases, administration of metoclopramide, a pharmaceutically-acceptable salt of metoclopramide, or a solvate of metoclopramide is at a dose ranging from at least about 1 mg/kg to at least about 2 mg/kg over a period of not less than 15 minutes.

In some embodiments, the 5-HT₃-targeting drug or 5-HT_{3A}-targeting drug is ondansetron, a pharmaceutically-acceptable salt of ondansetron, or a solvate of ondansetron and is available in several dosage forms (e.g., oral tablets, oral suspension, or injectable solution, which can be used alone or in combination), as approved by the FDA. Oral tablets may include an effective amount of ondansetron ranging from at least about 4 mg to at least about 24 mg, from at least about 4 mg to at least about 16 mg, from at least about 4 mg to at least about 8 mg, from at least about 8 mg to at least about 24 mg, from at least about 8 mg to at least about 16 mg, or from at least about 16 mg to at least about 24 mg. Oral suspensions may include an effective amount of ondansetron of at least about 4 mg/mL. Injectable solutions may include an effective amount of ondansetron of at least about 2 mg/mL. In some cases, administration of ondansetron, a pharmaceutically-acceptable salt of ondansetron, or a solvate of ondansetron is at a dose ranging from at least about 6 mg per day to at least about 96 mg per day. In some cases, administration of ondansetron, a pharmaceutically-acceptable salt of ondansetron, or a solvate of ondansetron is at a dose ranging from at least about 4 mg every 8 hours to at least about 32 mg every 8 hours.

In some embodiments, the 5-HT₃-targeting drug or 5-HT_{3A}-targeting drug is granisetron, a pharmaceutically-acceptable salt of granisetron, or a solvate of granisetron and is available in several dosage forms (e.g., oral tablets, injectable solution, transdermal patch, or subcutaneous injection, which can be used alone or in combination), as approved by the FDA. Oral tablets may include an effective amount of granisetron of at least about 1 mg. Injectable solutions may include an effective amount of granisetron ranging from at least about 0.1 mg/mL to at least about 4 mg/mL or from at least 1 mg/mL to at least 4 mg/mL. A transdermal patch may include an effective amount of granisetron of 3.1 mg, which is released over a 24 hour period. A subcutaneous injection may include an effective amount of at least about 25 mg/mL. In some cases, administration of granisetron, a pharmaceutically-acceptable salt of granisetron, or a solvate of granisetron is at a dose ranging from at least about 2 mg per day to at least about 10 mg per day. In some cases, administration of granisetron, a pharmaceutically-acceptable salt of granisetron, or a solvate of granisetron by infusion is at a dose of at least about 10 mg/kg.

In some embodiments, the 5-HT₃-targeting drug or 5-HT_{3A}-targeting drug is dolasetron, a pharmaceutically-acceptable salt of dolasetron, or a solvate of dolasetron and is available in several dosage forms (e.g., oral tablets or injectable solution, which can be used alone or in combination), as approved by the FDA. Oral tablets may include an effective amount of dolasetron ranging from at least about 50 mg to at least about 100 mg. An injectable solution may include an effective amount of dolasetron of at least about 20 mg/mL. In some cases, administration of dolasetron, a pharmaceutically-acceptable salt of dolasetron, or a solvate of dolasetron is at a dose ranging from at least about 1.8 mg/kg to at least about 100 mg/kg.

In some embodiments, the 5-HT₃-targeting drug or 5-HT_{3A}-targeting drug is palonosetron, a pharmaceutically-acceptable salt of palonosetron, or a solvate of palonosetron and is available in several dosage forms (e.g., oral capsules or intravenous injectable solution, which can be used alone or in combination), as approved by the FDA. Oral capsules may include an effective amount of palonosetron ranging from at least about 0.25 mg to at least about 0.5 mg. Injectable intravenous solutions may include an effective amount of palonosetron of at least about 0.05 mg/mL. In some cases, administration of palonosetron, a pharmaceutically-acceptable salt of palonosetron, or a solvate of palonosetron is at a dose ranging from at least about 0.075 mg per day to at least about 0.25 mg per day.

In some embodiments, the 5-HT₃-targeting drug or 5-HT_{3A}-targeting drug is alosetron, a pharmaceutically-acceptable salt of alosetron, or a solvate of alosetron and is available as oral tablets, as approved by the FDA. Oral tablets may include an effective amount of alosetron ranging from at least about 0.5 mg to at least about 1 mg. In some cases, administration of alosetron, a pharmaceutically-acceptable salt of alosetron, or a solvate of alosetron is at a dose ranging from at least about 0.5 mg per day to at least about 2 mg per day or at least about 1 mg per day to at least about 2 mg per day.

In some embodiments, the 5-HT₃-targeting drug or 5-HT_{3A}-targeting drug is cilansetron, a pharmaceutically-acceptable salt of cilansetron, or a solvate of cilansetron. In some cases, administration of cilansetron, a pharmaceutically-acceptable salt of cilansetron, or a solvate of cilansetron is given orally at a dose of at least about 6 mg per day.

In some embodiments, the 5-HT₃-targeting drug or 5-HT_{3A}-targeting drug is tropisetron, a pharmaceutically-acceptable salt of tropisetron, or a solvate of tropisetron. Tropisetron may be administered by intravenous injection or oral tablets. Injectable intravenous solutions may include an effective amount of tropisetron of at least about 1 mg/mL and oral tablets may include an effective amount of tropisetron of at least 5 mg. In some cases, administration of tropisetron, a pharmaceutically-acceptable salt of tropisetron, or a solvate of tropisetron is at a dose ranging from at least about 2 mg per day to at least about 5 mg per day.

In some embodiments, the 5-HT₃-targeting drug or 5-HT_{3A}-targeting drug is ramosetron, a pharmaceutically-acceptable salt of ramosetron, or a solvate of ramosetron and is available as an oral tablet or injectable solution. Oral tablets may include an effective amount of ramosetron of at least about 150 micrograms (μg). Injectable solutions may include an effective amount of ramosetron of at least about 150 μg per mL (μg/mL). In some cases, administration of ramosetron, a pharmaceutically-acceptable salt of ramosetron, or a solvate of ramosetron is at a dose ranging from at least about 5 μg per day to at least about 600 μg per day, from at least about 5 μg per day to at least about 300 μg per day, or from at least about 5 μg per day to at least about 100 μg per day.

Tubocurarine, clozapine, aripiprazole, olanzapine, metoclopramide, ondansetron, granisetron, dolasetron, palonosetron, alosetron, cilansetron, tropisetron, ramosetron, or a pharmaceutically-acceptable salt or solvate thereof may be used to combination with warfarin, Lisinopril, heparin, magnesium sulfate, haloperidol, glucagon, metoprolol, furosemide, hydralazine, or a pharmaceutically-acceptable salt or solvate of any of the preceding.

Warfarin is available as oral tablets, as approved by the FDA. Oral tablets may include an effective amount of warfarin ranging from at least about 1 mg to at least about 25 mg, from at least about 1 mg to at least about 10 mg, from at least about 1 mg to at least about 7.5 mg, from at least about 1 mg to at least about 6 mg, from at least about 1 mg to at least about 5 mg, from at least about 1 mg to at least about 3 mg, from at least about 1 mg to at least about 2.5 mg, from at least about 2 mg to at least about 25 mg, from at least about 2 mg to at least about 10 mg, from at least about 2 mg to at least about 7.5 mg, from at least about 2 mg to at least about 6 mg, from at least about 2 mg to at least about 5 mg, from at least about 2 mg to at least about 3 mg, from at least about 3 mg to at least about 25 mg, from at least about 3 mg to at least about 10 mg, from at least about 3 mg to at least about 7.5 mg, from at least about 3 mg to at least about 6 mg, from at least about 3 mg to at least about 5 mg, from at least about 4 mg to at least about 25 mg, from at least about 4 mg to at least about 10 mg, from at least about 4 mg to at least about 7.5 mg, from at least about 4 mg to at least about 6 mg, from at least about 5 mg to at least about 25 mg, from at least about 5 mg to at least about 10 mg, from at least about 5 mg to at least about 7.5 mg, from at least about 6 mg to at least about 25 mg, from at least about 6 mg to at least about 10 mg, from at least about 6 mg to at least about 7.5 mg, from at least about 7.5 mg to at least about 25 mg, from at least about 7.5 mg to at least about 10 mg, or from at least about 10 mg to at least about 25 mg. In some cases, warfarin is administered at a dose ranging from at least about 2 mg per day to at least 10 mg per day with ondansetron, a pharmaceutically-acceptable salt of ondansetron, or a solvate of ondansetron at a dose ranging from at least about 6 mg per day to at least about 96 mg per day or at a dose ranging from at least about 4 mg every 8 hours to at least about 32 mg every 8 hours.

Lisinopril, a pharmaceutically-acceptable salt of Lisinopril, or a solvate of Lisinopril is available as oral tablets, as approved by the FDA. Oral tablets may include an effective amount of Lisinopril ranging from at least about 2 mg to at least about 40 mg, from at least about 2.5 mg to at least about 40 mg, from at least about 2.5 mg to at least about 30 mg, from at least about 2.5 mg to at least about 20 mg, from at least about 2.5 mg to at least about 10 mg, from at least about 2.5 mg to at least about 5 mg, from at least about 5 mg to at least about 40 mg, from at least about 5 mg to at least about 30 mg, from at least about 5 mg to at least about 20 mg, from at least about 5 mg to at least about 10 mg, from at least about 10 mg to at least about 40 mg, from at least about 10 mg to at least about 30 mg, from at least about 10 mg to at least about 20 mg, from at least about 20 mg to at least about 40 mg, from at least about 20 mg to at least about 30 mg, or from at least about 30 mg to at least about 40 mg. In some cases, Lisinopril, a pharmaceutically-acceptable salt of Lisinopril, or a solvate of Lisinopril is administered at a dose ranging from at least about 5 mg per day to at least 80 mg per day with ondansetron, a pharmaceutically-acceptable salt of ondansetron, or a solvate of ondansetron at a dose ranging from at least about 6 mg per day to at least about 96 mg per day or at a dose ranging from at least about 4 mg every 8 hours to at least about 32 mg every 8 hours.

Heparin, a pharmaceutically-acceptable salt of heparin, or a solvate of heparin is available in several dosage forms (e.g., injectable solution, intravenous injection, or subcutaneous injection), as approved by the FDA. Heparin may be administered by continuous intravenous infusion or intermittent intravenous injection. Injectable solutions of heparin may include an effective amount of heparin ranging from at least 1,000 units per mL (units/mL) to at least about 20,000 units/mL, from at least about 1,000 units/mL to at least about 10,000 units/mL, from at least about 1,000 units/mL to at least about 5,000 units/mL, from at least about 5,000 units/mL to at least about 20,000 units/mL, from at least about 5,000 units/mL to at least about 10,000 units/mL, or from at least about 10,000 units/mL to at least about 20,000 units/mL. In some cases, heparin, a pharmaceutically-acceptable salt of heparin, or a solvate of heparin is administered at a dose ranging from at least about 20,000 units to at least about 40,000 units by continuous intravenous infusion per day or at a dose ranging from at least about 5,000 units to at least about 10,000 units by intermittent intravenous injection every 4 to 6 hours with ondansetron, a pharmaceutically-acceptable salt of ondansetron, or a solvate of ondansetron at a dose ranging from at least about 6 mg per day to at least about 96 mg per day or at a dose ranging from at least about 4 mg every 8 hours to at least about 32 mg every 8 hours.

Magnesium sulfate, a pharmaceutically-acceptable salt of magnesium sulfate, or a solvate of magnesium sulfate is available in several dosage forms (e.g., oral tablets, injectable solution, intravenous injection, or intramuscular injection), as approved by the FDA. Injectable solutions may include magnesium sulfate in an effective amount ranging from at least about 10 mg/mL to at least about 80 mg/mL, from at least about 10 mg/mL to at least about 40 mg/mL, from at least about 10 mg/mL to at least about 20 mg/mL, from at least about 20 mg/mL to at least about 80 mg/mL, from at least about 20 mg/mL to at least about 40 mg/mL, from at least about 40 mg/mL to at least about 80 mg/mL. Intramuscular injections may include magnesium sulfate in an effective amount of at least about 500 mg/mL. In some cases, magnesium sulfate is administered at a dose of at least about 1 gram by intramuscular injection every 6 hours or at a dose ranging from at least about 30 mg/kg d to at least about 60 mg/kg d with ondansetron, a pharmaceutically-acceptable salt of ondansetron, or a solvate of ondansetron at a dose ranging from at least about 6 mg per day to at least about 96 mg per day or at a dose ranging from at least about 4 mg every 8 hours to at least about 32 mg every 8 hours.

Haloperidol, a pharmaceutically-acceptable salt of haloperidol, or a solvate of haloperidol is available in several dosage forms (e.g. oral tablets, injectable solution, or oral suspension), as approved by the FDA. Oral tablets may include haloperidol in an effective amount ranging from at least about 0.5 mg to at least about 20 mg, from at least about 0.5 mg to at least about 10 mg, from at least about 0.5 mg to at least about 5 mg, from at least about 0.5 mg to at least about 2 mg, from at least about 0.5 mg to at least about 1 mg, from at least about 1 mg to at least about 20 mg, from at least about 1 mg to at least about 10 mg, from at least about 1 mg to at least about 5 mg, from at least about 1 mg to at least about 2 mg, from at least about 2 mg to at least about 20 mg, from at least about 2 mg to at least about 10 mg, from at least about 2 mg to at least about 5 mg, from at least about 5 mg to at least about 20 mg, from at least about 5 mg to at least about 10 mg, or from at least about 10 mg to at least about 20 mg. Injectable solutions may include haloperidol in an effective amount ranging from at least about 1 mg/mL to at least about 100 mg/mL, from at least about 1 mg/mL to at least about 50 mg/mL, from at least about 1 mg/mL to at least about 5 mg/mL, from at least about 5 mg/mL to at least about 100 mg/mL, from at least about 5 mg/mL to at least about 50 mg/mL, or from at least about 50 mg/mL to at least about 100 mg/mL. Oral suspensions may include haloperidol in an effective amount of at least about 2 mg/mL. In some cases, haloperidol, a pharmaceutically-acceptable salt of haloperidol, or a solvate of haloperidol is administered as an oral tablet at a dose ranging from at least about 1 mg per day to at least about 100 mg per day or as an injectable solution at a dose ranging from at least about 2 mg to at least about 5 mg by intramuscular injection every 4 to 8 hours with ondansetron, a pharmaceutically-acceptable salt of ondansetron, or a solvate of ondansetron at a dose ranging from at least about 6 mg per day to at least about 96 mg per day or at a dose ranging from at least about 4 mg every 8 hours to at least about 32 mg every 8 hours.

Glucagon, a pharmaceutically-acceptable salt of glucagon, or a solvate of glucagon is available in several dosage forms (e.g., injectable solution, subcutaneous injection, or a nasal powder), as approved by the FDA. Injectable solutions may include glucagon in an effective amount of from at least about 1 mg per vial. Subcutaneous injection solutions may include glucagon in an effective amount ranging from at least about 1 mg/mL to at least about 5 mg/mL. In some cases, glucagon, a pharmaceutically-acceptable salt of glucagon, or a solvate of glucagon is administered as an injectable solution intravenously at a dose ranging at least about 0.2 mg to at least about 0.75 mg or as an injectable solution intramuscularly at a dose ranging from at least about 1 mg to at least about 2 mg with ondansetron, a pharmaceutically-acceptable salt of ondansetron, or a solvate of ondansetron at a dose ranging from at least about 6 mg per day to at least about 96 mg per day or at a dose ranging from at least about 4 mg every 8 hours to at least about 32 mg every 8 hours.

Metoprolol, a pharmaceutically-acceptable salt of metoprolol, or a solvate of metoprolol is available in several dosage forms (e.g., oral tablets, oral capsules, or injectable solution, which can be used alone or in combination), as approved by the FDA. Oral tablets and oral capsules may include metoprolol in an effective amount ranging from at least about 25 mg to at least about 200 mg, from at least about 25 mg to at least about 100 mg, from at least about 25 mg to at least about 75 mg, from at least about 25 mg to at least about 50 mg, from at least about 25 mg to at least about 37.5 mg, from at least about 37.5 mg to at least about 200 mg, from at least about 37.5 mg to at least about 100 mg, from at least about 37.5 mg to at least about 75 mg, from at least about 37.5 mg to at least about 50 mg, from at least about 50 mg to at least about 200 mg, from at least about 50 mg to at least about 100 mg, from at least about 50 mg to at least about 100 mg, from at least about 75 mg to at least about 200 mg, from at least about 75 mg to at least about 100 mg, or from at least about 100 mg to at least about 200 mg. Injectable solutions may include metoprolol in an effective amount of at least about 1 mg/mL. In some cases, metoprolol, a pharmaceutically-acceptable salt of metoprolol, or a solvate of metoprolol is administered as an oral tablet or oral capsule at a dose ranging from at least about 100 mg per day to at least about 450 mg per day or as an injectable solution intravenously at a dose of at least about 5 mg every 2 hours for 3 total doses with ondansetron, a pharmaceutically-acceptable salt of ondansetron, or a solvate of ondansetron at a dose ranging from at least about 6 mg per day to at least about 96 mg per day or at a dose ranging from at least about 4 mg every 8 hours to at least about 32 mg every 8 hours.

Furosemide, a pharmaceutically-acceptable salt of furosemide, or a solvate of furosemide is available in several dosage forms (e.g., oral tablets, oral suspension, or injectable solution, which can be used alone or in combination), as approved by the FDA. Oral tablets may include furosemide in an effective amount ranging from at least about 20 mg to at least about 80 mg, from at least about 20 mg to at least about 40 mg, or from at least about 40 mg to at least about 80 mg. Oral suspensions may include furosemide in an effective amount ranging from at least about 8 mg/mL to at least about 10 mg/mL. Injectable solutions may include furosemide in an effective amount of at least about 10 mg/mL. In some cases, furosemide, a pharmaceutically-acceptable salt of furosemide, or a solvate of furosemide is administered as an oral tablet at a dose ranging from at least about 20 mg per day to at least about 120 mg per day or as an injectable solution intravenously at a dose ranging from at least about 20 mg to at least about 40 mg over 1 to 2 minutes once or twice daily or an injectable solution intramuscularly at a dose ranging from at least about 20 mg to at least about 40 mg once or twice daily with ondansetron, a pharmaceutically-acceptable salt of ondansetron, or a solvate of ondansetron at a dose ranging from at least about 6 mg per day to at least about 96 mg per day or at a dose ranging from at least about 4 mg every 8 hours to at least about 32 mg every 8 hours.

Hydralazine, a pharmaceutically-acceptable salt of hydralazine, or a solvate of hydralazine is available in several dosage forms (e.g. oral tablets, oral capsules, or injectable solution, which can be used alone or in combination), as approved by the FDA. Oral tablets and oral capsules may include hydralazine in an effective amount ranging from at least about 10 mg to at least about 100 mg, from at least about 10 mg to at least about 50 mg, from at least about 10 mg to at least about 25 mg, from at least about 25 mg to at least about 100 mg, from at least about 25 mg to at least about 50 mg, or from at least about 50 mg to at least about 100 mg. Injectable solutions may include hydralazine in an effective amount of at least about 20 mg/mL. In some cases, hydralazine, a pharmaceutically-acceptable salt of hydralazine, or a solvate of hydralazine is administered as an oral tablet at a dose ranging from at least about 40 mg per day to at least about 200 mg per day or as an intravenous bolus at a dose ranging from at least about 20 mg to at least about 40 mg or as an injectable solution intramuscularly at a dose ranging from at least about 20 mg to at least about 40 mg with ondansetron, a pharmaceutically-acceptable salt of ondansetron, or a solvate of ondansetron at a dose ranging from at least about 6 mg per day to at least about 96 mg per day or at a dose ranging from at least about 4 mg every 8 hours to at least about 32 mg every 8 hours.

### EXAMPLES

Multiple comorbidities of AKI have been shown by epidemiology studies including cancer, cardiovascular diseases, complex surgery, liver diseases, and diabetes mellitus. Discrepancy diseases that take part in AKI development lead to diversity of medication records. Given the prognosis and medical records of ICU patients with AKI, drugs that can have beneficial effects on preventing those patients from death can be determined. In this study two publicly available databases (MIMIC III and eICU) of electronic medical records (EMR) from more than twenty thousand AKI patients in ICU stays were analyzed. The focus was to determine associations between medication use and outcomes of ICU mortality rate.

### Example 1

Clinical electronical medical records (EMR) from patients with acute kidney injury (AKI) in the intensive care unit (ICU) were analyzed to identify medications with potentially beneficial effects on decreasing mortality in this patient population.

The Cox proportional hazards model was used to investigate the association between the survival time of AKI patients from the MIMIC III database and their medication use. The effects of drug(s) associated with lower death rates were then validated in the eICU database through Propensity Score Matching (PSM) to adjust baseline, followed by chi-square test to calculate the significance on preventing AKI patients from death. Gene Expression Signature (GES) was used to explore the molecular mechanism of the identified drug(s) on AKI.

### Methods

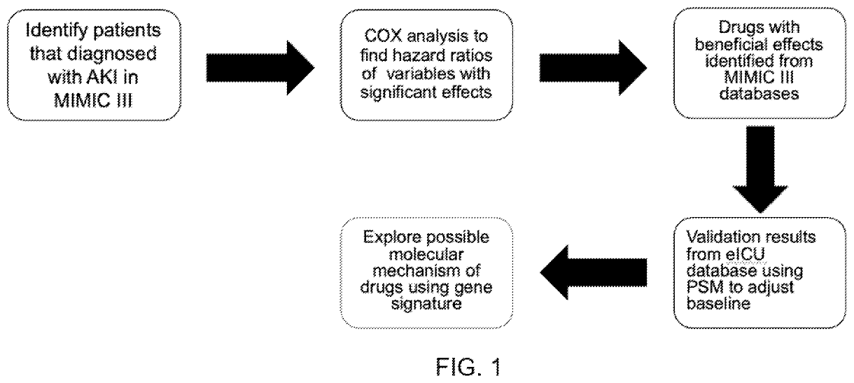

Overview of the research design: FIG. 1 shows a flow chart of the procedures. The AKI patients were first identified from the MIMIC III database with diagnosis codes and the clinical data was extracted. The COX proportional-hazards ratios was used to select factors which showed beneficial effects on preventing AKI patients from death in the ICU. Among these factors, drug(s) with potential beneficial effects were identified and a literature search was conducted to confirm our findings. Data from eICU database was then used to validate the effect of drug(s). Finally, gene expression signature (GES) was used to find the possible mechanism of these drug(s) for their beneficial effects on AKI.

Data source: The study is based on Multiparameter Intelligent Monitoring in Intensive Care III (MIMIC III) v1.4, which is the latest version of an openly available clinical database developed by the MIT Lab for Computational Physiology (Johnson, A. E. et al. “MIMIC-III, a freely accessible critical care database”, Scientific Data, 2016, 3, 1-9). This database is comprised of more than 60,000 ICU admissions in Beth Israel from June 2001 to October 2012 which includes laboratory tests, medication records, diagnoses, and more. To acquire access to MIMIC III, the CITI “Data or Specimens Only Research” course was completed (record ID: 36580723). To safeguard patient privacy, data is de-identified; therefore, informed consent was waived.

The second database, eICU, was used to further validate our findings (Pollard, T. J. et al. “The eICU Collaborative Research Database, a freely available multi-center database for critical care research” Scientific Data, 2018, 5, 180178). The eICU database is a collaborative research database which contains of over 200,000 multi-center critical care records in ICUs in the United States through 2014-2015 and was made available by Philips Healthcare in partnership with the MIT Laboratory for Computational Physiology. The CITI “Data or Specimens Only Research” course was also required for the access to this database. Data were de-identified to safeguard patient privacy; therefore, informed consent was also waived.

The Illumina BaseSpace software was used to explore possible molecular mechanisms of drugs that have potentially beneficial effect in AKI (Nakamura, K. et al. “Sequence-specific error profile of Illumina sequencers”, Nucleic acids research, 2011, 39, e90-e90). The BaseSpace software contains a number of applications which provide next-generation sequencing (NGS), transcriptional, and proteomic data analysis, mostly developed or optimized by Illumina.

Population selection criteria: The disease codes 584.5, 584.6, 584.7, 584.8, and 584.9 were used to search the diagnosis table in the MIMIC III database to identify AKI patients. For the eICU database, the keyword search (“acute renal failure”) was in the diagnosis table to identify AKI patients.

Data extraction: From the MIMIC III database, the demographic characteristics, physiological index, International Classification of Diseases (ICD-9) codes, medications, laboratory tests, and time events were extracted for these AKI patients. These variables were classified into three categories: first day vital information, medication use information, and other variables. The first day vital and lab test information included: mean oxygen saturation; mean temperature; mean respiration rate; mean potassium level; mean white blood cell count; mean prothrombin time; mean hematocrit level; mean creatinine level; mean hemoglobin level; mean platelet level; peripheral vascularity; mean blood pressure; mean partial thromboplastin time; mean lactic acid level; mean diastolic blood pressure; mean chloride level; mean sodium level; mean bicarbonate level; mean albumin level; mean systolic blood pressure; mean Glucose level; mean bands cell count; pulmonary circulation; mean level of bilirubin; mean interaction between the Cardiac Rapidly; mean heart rate; mean anion gap; mean blood urea nitrogen; Coagulopathy; liver disease; metastatic cancer; stroke; chronic pulmonary; lymphoma; alcohol abuse; Rheumatoid arthritis; renal failure; hypertension; congestive heart failure; solid tumor; gender; and age. Medication use information was filtrated to be the top 50 most commonly used drugs among these AKI patients and include: Morphine; Warfarin; Norepinephrine; Magnesium Sulfate; Fluconazole; Furosemide; Oxycodone; Haloperidol; Heparin; Metoclopramide; Metoprolol; Meropenem; Lisinopril; Propofol; Captopril; Amiodarone; Hydralazine; Acetaminophen; Piperacillin; Phytonadione; Lactulose; Ondansetron; Nitroglycerin; Fentanyl; Albuterol; Ipratropium; Bisacodyl; Tacrolimus; Prednisone; Senna; Aspirin; Cefepime; Hydromorphone; Insulin; Levofloxacin; Metronidazole; Lorazepam; Famotidine; Midazolam; Chlorhexidine; Dexamethasone; Diltiazem; Glucagon; Pantoprazole; Docusate; Vancomycin; Atorvastatin; and Levothyroxine. The other variables included: Sequential Organ Failure Assessment (SOFA) Score; Simplified Acute Physiology Score (SAPS) II; Elixhauser's Comorbidity Index; and KDIGO stages in the first 48 hours.

From the eICU database, APS scores (Pollack, M. M. et al. “The Pediatric Risk of Mortality III-Acute Physiology Score (PRISM III-APS): a method of assessing physiologic instability for pediatric intensive care unit patients”, The Journal of Pediatrics 1997, 131, 575-581), drug administrations, and patients' status on ICU discharge (alive or dead) were extracted.

Drug and disease-induced DEGs (differentially expressed genes) were extracted by using the Illumina BaseSpace software.

Cox analysis to identify factors that significantly influence the ICU morbidities of AKI patients: The extracted data were further analyzed with Cox proportional hazard model (CoxPH) to control for multiple variants and identify hazard ratios for single drug usage (Katzman, J. L. et al. “DeepSurv: personalized treatment recommender system using a Cox proportional hazards deep neural network”, BMC medical research methodology, 2018, 18, 24). A patient's death within 24 hours within their ICU discharge was considered as the event in the analysis and a patient's length of stay in ICU was considered as time to event. Eleven variables were dropped because their variances were too low for CoxPH analysis, including: cardiac arrhythmias, paralysis, hypothyroidism, peptic ulcer, obesity, weight loss, blood loss anemia, deficiency anemias, drug abuse, psychoses, and depression.

Validation of our findings from eICU database: The findings were further validated with the eICU database. The acute physiology score (APS), which can be used to predict the mortality risk, was extracted and matched by propensity score to adjust the baseline between patients who took a drug of interest during their ICU stay and patients who did not. The ICU death rates for patients on/not on a drug of interest by chi-square test were compared (Satorra, A. et al. “A scaled difference chi-square test statistic for moment structure analysis” Psychometrika 2001, 66, 507-514).

Investigation of possible molecular mechanisms by comparison of gene transcriptional profiles: To understand the molecular mechanism behind the beneficial effects of a drug of interest on AKI outcomes, the gene expression profiles induced by drugs identified in the analyses with lower ICU death rates were analyzed. Differentially expressed genes (DEGs) induced by drugs were collected from Illumina BaseSpace software. In BaseSpace, only genes with p values <0.05 and absolute fold changes greater than 1.2 were considered as DEGs. All the DEGs induced by a drug can be considered as a gene signature for this drug. The drug-induced gene expression datasets were selected by searching with drug names. The drug-induced DEGs were used to search against disease-induced DEGs to find potential associations between these drugs and kidney diseases through the commonly modulated genes. The molecular pathways of those common genes involved were collected to investigate the possible molecular mechanisms of beneficial effects. All BaseSpace analyses were performed using the default parameters.

Statistical analysis: The Cox analysis was performed by Python using lifelines survival regression package to measure the association between patient death time and variables.

The chi-square (χ²) test was performed to evaluate to difference of death rate between target medication users versus non-users. Propensity score matching (PSM), which was conducted by R package Match It, was used to balance the baseline condition of the patient of both groups. Student t-test was used to measure the mean difference between two samples. All statistical t-tests were calculated by R. The Running Fisher algorithm is used by Basespace software to assess the statistical significance of overlapping between two gene sets, where p-values are computed by a Fisher's exact test. A p<0.05 was used as the threshold for statistical significance for all analyses except where stated otherwise.

### Results and Discussion

Factors that significantly contribute to the ICU mortality of AKI patients by Cox analysis: From the MIMIC III database, 9,536 AKI patients were identified and among them, 9,443 patients had completed information of demographic, ICU stay, and the first day vital information. Patients with multiple ICU stay records were deleted to simplify the calculation, resulting in 7,313 unique patients and 1,661 of those patients died during their ICU stays (or ICU death rate: 22.7%). The information from those 7,313 patients was used to build a CoxPH model and was used to identify factors that contributed significantly to the prediction of death in ICU. Table 1 lists those factors with p values <0.05.

TABLE 1
Hazard Ratio and significance of variables

| | | Hazard | Standard | |
|---|---|---|---|---|
| | Coeffi- | Ratio | Deviation | |
| Variable | cient | (HR) | (SD) of HR | p-value |
| Morphine | 1.02 | 2.76 | 0.0643 | 3.24E−56 |
| Spo2 Mean | −0.0566 | 0.944 | 0.00503 | 1.93E−29 |
| Sapsii | 0.0234 | 1.02 | 0.00252 | 1.68E−20 |
| Norepinephrine | 0.566 | 1.76 | 0.0727 | 6.63E−15 |
| Warfarin | −1.03 | 0.357 | 0.1364 | 4.16E−14 |
| Magnesium Sulfate | −0.445 | 0.641 | 0.0641 | 3.62E−12 |
| Fluconazole | −0.601 | 0.548 | 0.0936 | 1.31E−10 |
| Furosemide | −0.413 | 0.662 | 0.0667 | 6.09E−10 |
| Oxycodone | −0.595 | 0.551 | 0.0978 | 1.15E−09 |
| Tempc Mean | −0.208 | 0.812 | 0.0347 | 2.00E−09 |
| Resprate Mean | 0.0351 | 1.04 | 0.00601 | 5.07E−09 |
| Haloperidol | −0.426 | 0.653 | 0.0850 | 5.52E−07 |
| Heparin | −0.322 | 0.725 | 0.0663 | 1.24E−06 |
| Metoclopramide | −0.403 | 0.668 | 0.0861 | 2.81E−06 |
| Potassium Avg | 0.209 | 1.23 | 0.0463 | 6.16E−06 |
| Metoprolol | −0.297 | 0.743 | 0.0676 | 1.11E−05 |
| Meropenem | −0.329 | 0.720 | 0.0763 | 1.61E−05 |
| Wbc Avg | 0.00653 | 1.01 | 0.00153 | 2.05E−05 |
| Pt Avg | 0.01785 | 1.02 | 0.00440 | 5.00E−05 |
| Lisinopril | −0.596 | 0.551 | 0.165 | 0.000311 |
| Propofol | −0.234 | 0.791 | 0.0687 | 0.00066 |
| Captopril | −0.481 | 0.618 | 0.142 | 0.00068 |
| Hematocrit Avg | 0.0602 | 1.06 | 0.0180 | 0.000847 |
| Amiodarone | 0.245 | 1.28 | 0.0745 | 0.000997 |
| Creatinine Avg | −0.0823 | 0.921 | 0.0254 | 0.00121 |
| Hydralazine | −0.283 | 0.753 | 0.0904 | 0.00174 |
| Acetaminophen | −0.191 | 0.826 | 0.0638 | 0.00276 |
| Piperacillin | −0.183 | 0.833 | 0.0635 | 0.00400 |
| Hemoglobin Avg | −0.150 | 0.861 | 0.0536 | 0.00490 |
| Phytonadione | −0.186 | 0.830 | 0.0667 | 0.00530 |
| Lactulose | −0.198 | 0.820 | 0.0718 | 0.00579 |
| Congestive Heart | −1.26 | 0.283 | 0.459 | 0.00594 |
| Failure | | | | |
| Sofa | −0.0305 | 0.970 | 0.0114 | 0.00758 |
| Platelet Avg | −0.00064 | 0.999 | 0.000242 | 0.00805 |
| Peripheral Vascular | −0.421 | 0.656 | 0.170 | 0.0130 |
| Meanbp Mean | −0.0171 | 0.983 | 0.00695 | 0.0142 |
| Ptt Avg | 0.00270 | 1.00 | 0.00123 | 0.0284 |
| Ondansetron | −0.190 | 0.827 | 0.0883 | 0.0312 |
| Nitroglycerin | 0.190 | 1.21 | 0.0918 | 0.0386 |
| Solid Tumor | 0.373 | 1.45 | 0.181 | 0.0391 |
| Fentanyl | 0.175 | 1.19 | 0.0867 | 0.0437 |
| Coagulopathy | 2.07 | 7.96 | 1.03 | 0.0438 |
| Lactate Avg | 0.0298 | 1.03 | 0.0152 | 0.0492 |

To determine the effect of all of the variables on patients' mortality through ICU admission and 24 hours after discharge, the Cox model was used to identify the Hazard Ratio (HR) of these variables among the 7,313 patients. HRs demonstrate two sides of effect, positive and negative. A negative coefficient forced HR values away from 1 to 0 to show protective effects of these variables, which means preventing patients from mortality. A positive coefficient value forced HR values to be higher than 1 to illustrate ‘promoting effect’ on patient death of these variables.

Drugs potentially have beneficial effects on AKI patients' mortality in ICU: A literature search was conducted and it was found that among those drugs identified in Table 1, most of the positive or negative effects of these drugs on all ICU patients through mortality have been reported in literatures and/or clinical trials before (Table 2). In Table 2, Y has clinical trials and/or literature support and N has no clinical trials and/or literature support.

TABLE 2
Medications identified with significant effects on mortality in ICU for AKI patients

| | | Reported for | Reported for | |
|---|---|---|---|---|
| | Protecting | protecting | protecting | |
| | effect | effect in | effect in | |
| | from | all ICU | ICU patients | |
| Medication | death | patients | with AKI | Medical use of drug |
| Morphine | N | Y¹ | N | Treatment of moderate to |
| | | | | severe pain |
| Norepinephrine | N | Y² | N | Treatment of low blood |
| | | | | pressure and heart failure |
| Warfarin | Y | Y¹ | N | Treatment and prevention |
| | | | | blood clots |
| Magnesium | Y | Y³ | Y⁴ | Prevention and control of |
| Sulfate | | | | seizures in preeclampsia and |
| | | | | eclampsia |
| Fluconazole | Y | Y⁵ | Y⁶ | Treatment and prevention of |
| | | | | fungal infections |
| Furosemide | Y | Y⁷ | Y⁸ | Treatment of fluid retention |
| | | | | (edema) and swelling caused |
| | | | | by congestive heart failure, |
| | | | | liver disease, kidney disease, |
| | | | | and other medical conditions |
| Oxycodone | Y | Y⁹ | N | Treatment of moderate to |
| | | | | severe pain |
| Haloperidol | Y | N | N | Antipsychotic |
| Heparin | Y | Y¹⁰ | N | Anticoagulant, also used to |
| | | | | treat heart attacks and |
| | | | | unstable angina |
| Metoclopramide | Y | N | N | Antiemetic and gut motility |
| | | | | stimulator |
| Metoprolol | Y | Y¹¹ | N | Beta Blocker |
| Meropenem | Y | Y¹² | Y¹³ | Antibiotic |
| Lisinopril | Y | Y¹⁴ | Y¹⁵ | ACE inhibitor, can be used to |
| | | | | treat high blood pressure and |
| | | | | heart failure |
| Propofol | Y | Y¹⁶ | Y¹⁷ | Anesthetic |
| Captopril | Y | Y¹⁸ | Y¹⁹ | ACE inhibitor; can also be |
| | | | | used to treat kidney problems |
| | | | | caused by diabetes |
| Amiodarone | N | N | N | Treatment of heart rhythm |
| | | | | problems |
| Hydralazine | Y | Y | Y | Treatment of high blood |
| | | | | pressure |
| Acetaminophen | Y | N | N | Analgesic |
| Piperacillin | Y | Y | N | Penicillin Antibiotic |
| Phytonadione | Y | Y | N | Helps blood clot |
| Lactulose | Y | N | N | Treatment of constipation; |
| | | | | can also treat liver disease |
| Ondansetron | Y | N | N | Prevention of nausea and |
| | | | | vomiting |
| Nitroglycerin | N | N | N | Vasodilator |
| Fentanyl | N | Y | N | Narcotic, treatment for |
| | | | | severe pain |
| Hydromorphone | Y | N | N | Treatment of moderate to |
| | | | | severe pain |
| Docusate | Y | N | N | Treatment of constipation |
| Pantoprazole | Y | N | N | Treatment of stomach |
| | | | | ulcers, erosive esophagitis |
| | | | | due to gastroesophageal |
| | | | | reflux disease |
| Midazolam | N | Y²⁰ | N | Used for anesthesia, |
| | | | | procedural sedation, trouble |
| | | | | sleeping, and severe |
| | | | | agitation |
| Lorazepam | N | N | N | Treatment of anxiety |
| | | | | disorders, trouble sleeping, |
| | | | | active seizures, and |
| | | | | chemotherapy induced |
| | | | | nausea and vomiting |

¹Beard Jr., E. L. “The American society of health system pharmacists”, JONA'S Healthcare Law, Ethics and Regulation, 2001, 3, 78-79.
²Rang, H. et al. “Chapter 14: Noradrenergic transmission.” Rang & Dale's Pharmacology, Elsevier Health Sciences, 2014, 177-196.
³Pryde, P. G. et al. “Contemporary usage of obstetric magnesium sulfate: indication, contraindication, and relevance of dose”, Obstetrics & Gynecology, 2009, 114, 669-673.
⁴Firouzi, A. et al. “Intravenous magnesium sulfate: new method in prevention of contrast-induced nephropathy in primary percutaneous coronary intervention”, International Urology and Nephrology, 2015, 47, 521-525.
⁵Seward, H. E. et al. “Crystal structure of the Mycobacterium tuberculosis P450 CYP121-fluconazole complex reveals new azole drug-P450 binding mode”, Journal of Biological Chemistry, 2006, 281, 39437-39443.
⁶Sinnollareddy, M. G. et al. “Pharmacokinetics of fluconazole in critically ill patients with acute kidney injury receiving sustained low-efficiency diafiltration” International Journal of Antimicrobial Agents, 2015, 45, 192-195.
⁷Buggey, J. et al. “A reappraisal of loop diuretic choice in heart failure patients”, American Heart Journal, 2015, 169, 323-333.
⁸Phakdeekitcharoen, B. et al. “The added-up albumin enhances the diuretic effect of furosemide in patients with hypoalbuminemic chronic kidney disease: a randomized controlled study” BMC Nephrology, 2012, 13, 92.
⁹Moradi, M. et al. “Use of oxycodone in pain management”, Anesthesiology and Pain Medicine, 2012, 1, 262.
¹⁰Agnelli, G. et al. “Enoxaparin plus compression stockings compared with compression stockings alone in the prevention of venous thromboembolism after elective neurosurgery”, New England Journal of Medicine, 1998, 339, 80-85.
¹¹Mohan, J. C. et al. “Rediscovering Chirality - Role of S-Metoprolol in Cardiovascular Disease Management”, J Assoc Physicians India, 2017, 65, 74-79Jagdish.
¹²Cho, J. C. et al. “Meropenem/Vaborbactam, the First Carbapenem/β-Lactamase Inhibitor Combination”, Ann Pharmacother, 2018, 52, 769-779.
¹³Dhillon, S. “Meropenem/Vaborbactam: A Review in Complicated Urinary Tract Infections”, 2018, Drugs, 78, 1259-1270.
¹⁴in LiverTox: Clinical and Research Information on Drug-Induced Liver Injury, National Institute of Diabetes and Digestive and Kidney Diseases, 2012.
¹⁵Sadat-Ebrahimi, S. R. et al. “An evidence-based systematic review of the off-label uses of Lisinopril”, Br J Clin Pharmacol, 2018, 84, 2502-2521.
¹⁶Walsh, C. T. “Propofol: Milk of Amnesia”, Cell, 2018, 175, 10-13.
¹⁷Zheng, G. et al. “Propofol attenuates sepsis-induced acute kidney injury by regulating miR-290-5p/CCL-2 signaling pathway”, Braz J Med Biol Res, 2018, 51, e7655.
¹⁸Rosendorff, C. “Captopril--an overview”, S Afr Med J, 1982, 62, 593-599.
¹⁹Garunk\check{s}tienė, R. et al. “Acute kidney injury in an extremely low birth weight infant with nephrolithiasis: a case report”, Acta Med Litu, 2018, 25, 166-172.
²⁰Ozdemir, D., et al., “Efficacy of continuous midazolam infusion and mortality in childhood refractory generalized convulsive status epilepticus”, Seizure, 2005, 14(2): 129-132.

Drugs reported to have beneficial effects on patient's survival in ICU include: diuretic drug (furosemide), anti-heart failure drugs (norepinephrine, heparin, metoprolol, lisinopril, captopril), and anti-infection drugs (fluconazole, meropenem, piperacillin). These drugs can reduce the risk of death in ICU, which gives support on protecting effects of other drugs without literature support.

A literature search was conducted on drugs to determine whether they were reported to have beneficial effects on AKI patients' recovery through death (Table 2). There were 8 of 20 drugs with beneficial effects (Table 1) reported by literature or clinical trials to have beneficial effect on AKI recovering, which may validate the effectiveness of other drugs. Through all drugs, ondansetron was noticed for its use for preventing nausea and vomiting, which seemed to be less related to preventing death and AKI recovery. The preventing effect of ondansetron on AKI patients' mortality was validated from other databases.

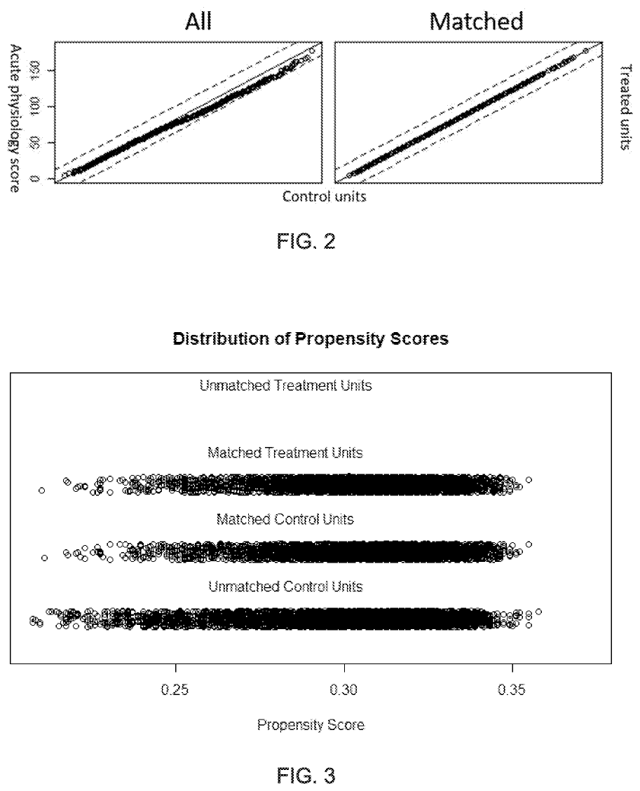

Validation of beneficial effects of ondansetron from the eICU database: 12,676 AKI patients having APS scores were identified from the eICU database. The mean values of APS scores from patients who took/did not take ondansetron are significantly different according to t-test. After the PSM procedure of a ratio of 1:1 matching (3,848:3,848 patients were matched), the APS scores between the two group are nearly the same with a t-test p-value around 1. Detailed information of the samples before and after PSM is shown in Table 3, and the APS score and propensity score tendency of the two samples are shown in FIGS. 2 and 3.

TABLE 3
The APS scores and statistical variables of patients
took/not took ondansetron before and after PSM.

| | Non-Ondansetron | | Ondansetron | | |
|---|---|---|---|---|---|
| | APS | | APS | | |
| | (mean (SD)) | N | (mean (SD)) | N | p-value |
| Before PSM | 61.12 (28.58) | 8,828 | 57.98 (25.69) | 3,848 | <0.001 |
| After PSM | 57.98 (25.69) | 3,848 | 57.98 (25.69) | 3,848 | 0.999 |

After the baseline APS adjustment by PSM, a lower death rate (12.45%) from the ondansetron group than the non-ondansetron control group (14.58%) was observed. The chi-square test p-value between the two groups was 0.007 which indicated that ondansetron does have an effect on lowering the mortality of AKI patients. Detailed information is shown in Table 4.

TABLE 4
The contingency table of the death event occurred
in patients took/not took ondansetron. p-value
is calculated according to chi-square test.

| | Non-Ondansetron | Ondansetron | Total | p-value |
|---|---|---|---|---|
| Death | 561 | 479 | 1,040 | 0.007 |
| Alive | 3,287 | 3,369 | 6,656 | |
| Total | 3,848 | 3,848 | 7,696 | |

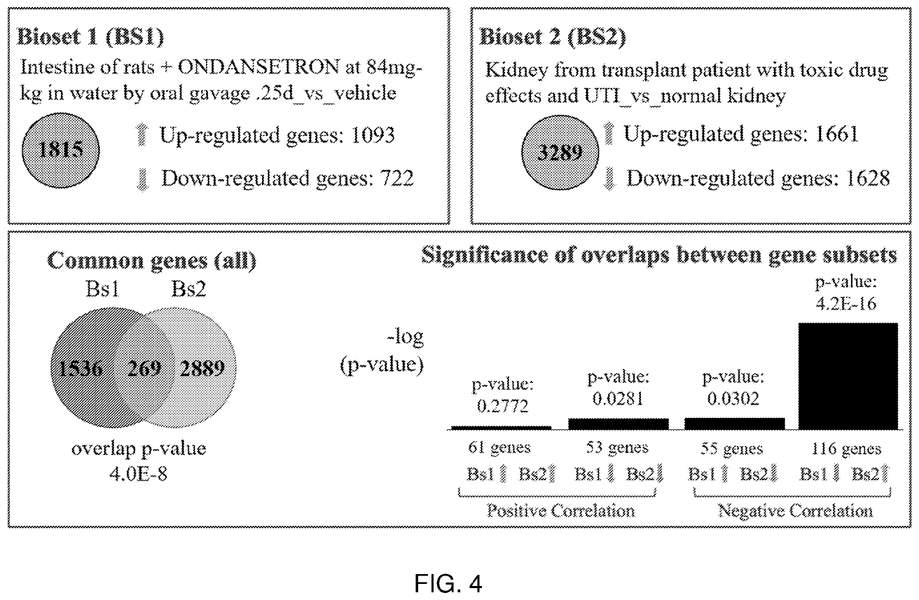

Molecular mechanism study by gene expression signatures: The gene expression profiles induced by ondansetron and AKI were further analyzed. Through the Basespace database, one ondansetron dataset from Chemical Effects in Biological Systems (CEBS) database was found (Waters, M. et al. “CEBS—Chemical Effects in Biological Systems: a public data repository integrating study design and toxicity data with microarray and proteomics data”, Nucleic Acids Research, 2007, 36, D892-D900) where intestines from rats were treated with ondansetron in vivo and assayed for expression. The “Intestine of rats+ONDANSETRON at 84 mg-kg in water by oral gavage 0.25d_vs_vehicle” bioset was selected to mimic the acute effects of this drug. This gene expression profile contains 1,093 up-regulated genes and 722 down-regulated genes. Searching with this GES, three biosets (Sarwal, M. et al. “Molecular heterogeneity in acute renal allograft rejection identified by DNA microarray profiling”, New England Journal of Medicine, 2003, 349, 125-138) of AKI from transplant patient with toxic drug effects with overlap p-values of 4.0E-08, 9.8E-07 and 4.9E-06, respectively, were identified (Table 5). Here UTI stands for urinary tract infection, and ARII stands for acute rejection type II. A detailed comparison is provided in FIG. 4.

TABLE 5
Comparison of ondansetron-induced gene expression
profiles with three AKI gene expression profiles

| | | | Overlap | Common |
|---|---|---|---|---|
| Biosets | Bioset Name | Genes | p-value | Genes |
| Ondansetron | Intestine of rats + ONDANSETRON at 84 mg-kg | 1,815 | — | — |
| | in water by oral gavage .25d_vs_vehicle (GEO | | | |
| | ID: GSE59927) | | | |
| AKI1 | Kidney from transplant patient with toxic drug | 3,289 | 4.0E−08 | 269 |
| | effects and UTI_vs_normal kidney (GEO ID: | | | |
| | GSE362) | | | |
| AKI2 | Kidney from transplant patient with toxic drug | 4,417 | 9.8E−07 | 345 |
| | effects_vs_normal kidney (GEO ID: GSE409) | | | |
| AKI3 | Kidney from transplant patient with toxic drug | 4,695 | 4.9E−06 | 345 |
| | effects, UTI, and ARII_vs_normal kidney (GEO | | | |
| | ID: GSE410) | | | |

To investigate the possible molecular mechanism of ondansetron on modulating AKI related molecular pathways, a meta-analysis on these four biosets was completed. From this analysis, it was found that Pathways in cancer, microRNA target genes by miR381, miR200b, miR101, and miR26, were down-regulated (Table 6). Furthermore, miR381 was reported to play a role in rat models of renal ischemia reperfusion injury (Zheng, G. H. et al. “MicroRNA-381-induced down-regulation of CXCR4 promotes the proliferation of renal tubular epithelial cells in rat models of renal ischemia reperfusion injury” Journal of Cellular Biochemistry, 2018, 119, 3149-3161. Target genes miR200b, miR101, and miR26 can be used as biomarkers for AKI (Kito, N. et al. “miRNA profiles of tubular cells: diagnosis of kidney injury”, BioMed Research International, 2015, 2015; Aguado-Fraile, E. et al. “A pilot study identifying a set of microRNAs as precise diagnostic biomarkers of acute kidney injury”, PLoS One, 2015, 10, e0127175). Cancer-related genes were down-regulated by ondansetron. As shown in Table 7, among those genes, Rela and Jak1 are the key proteins in the NF-KB pathway and JAK-STAT pathway, respectively, and ondansetron can down-regulate these two genes. In comparison, in AKI, these two genes are up-regulated. Inhibitors for those two pathways have been reported to have beneficial effects for AKI (Si, Y. et al. “Dexmedetomidine protects against renal ischemia and reperfusion injury by inhibiting the JAK/STAT signaling activation”, Journal of Translational Medicine, 2013, 11, 141; Ozkok, A. et al. “NF-κB transcriptional inhibition ameliorates cisplatin-induced acute kidney injury (AKI)”, Toxicology letters, 2016, 240, 105-113). Pld1 was upregulated in chronic kidney disease (CKD) and also here in the AKI biosets. PLD1 inhibitor can prevent vascular calcification in CKD patients (Skafi, N. et al. “Phospholipase D: A new mediator during high phosphate-induced vascular calcification associated with chronic kidney disease”, Journal of Cellular Physiology, 2019, 234, 4825-4839). Ondansetron can hold the key proteins which were not in active states to response to injury instantly and the consequences of damages caused by those responses might be mitigated.

TABLE 6
Enriched pathways in the Ondansetron and AKI biosets

| | | Ondansetron | AKI1 | AKI2 | AKI3 |
|---|---|---|---|---|---|
| Pathways | Genes | (p-value) | (p-value) | (p-value) | (p-value) |
| Pathways in cancer | 311 | 32 (2.1E−17) | 66 (5.6E−30) | 89 (5.8E−40) | 82 (5.7E−36) |
| Predicted Gene | 472 | 42 (4.3E−20) | 83 (2.2E−30) | 103 (1.9E−34) | 87 (6.2E−26) |
| Targets for miR- | | | | | |
| 381 | | | | | |
| Predicted Gene | 428 | 34 (6.8E−15) | 75 (2.9E−27) | 101 (1.1E−36) | 86 (1.7E−28) |
| Targets for miR- | | | | | |
| 200b | | | | | |
| Positive regulation | 482 | 39 (2.7E−17) | 67 (7.8E−21) | 89 (4.2E−28) | 92 (4.7E−31) |
| of cell | | | | | |
| differentiation | | | | | |
| Predicted Gene | 386 | 24 (1.7E−8) | 69 (3.0E−26) | 94 (5.0E−36) | 75 (8.3E−24) |
| Targets for miR- | | | | | |
| 101 | | | | | |
| Kinase binding | 426 | 41 (3.7E−21) | 56 (4.7E−18) | 77 (7.7E−26) | 79 (2.7E−28) |
| Targets of | 448 | 32 (1.1E−12) | 77 (4.1E−27) | 93 (1.4E−29) | 80 (1.3E−23) |
| MicroRNA | | | | | |
| CAGTATT, MIR- | | | | | |
| 200B, MIR-200C, | | | | | |
| MIR-429 | | | | | |
| Predicted Gene | 448 | 36 (8.6E−16) | 71 (1.5E−23) | 93 (3.1E−29) | 83 (3.2E−24) |
| Targets for miR-26 | | | | | |
| Response to | 326 | 31 (5.6E−17) | 59 (5.1E−22) | 86 (5.2E−33) | 65 (2.0E−20) |
| peptide hormone | | | | | |
| stimulus | | | | | |
| Genes involved in | 420 | 42 (4.3E−21) | 73 (3.6E−26) | 80 (1.5E−22) | 77 (1.5E−21) |
| Hemostasis | | | | | |

TABLE 7
Detailed gene expression fold changes
in ondansetron and AKI biosets

| Gene | Ondansetron | AKI1 | AKI2 | AKI3 |
|---|---|---|---|---|
| Rela | −265 | 1.37 | — | 1.64 |
| Jak1 | −10.7 | 1.74 | 0.65 | 0.36 |
| Ctbp2 | −10.3 | — | — | — |
| Fn1 | −10.3 | — | — | — |
| Rb1 | −9.02 | — | 1.51 | 1.48 |
| Ctnnb1 | −6.88 | −2.1 | −2.1 | −2.42 |
| Pld1 | −5.95 | — | 1.52 | 1.67 |
| Mapk9 | −5.48 | 1.29 | −1.8 | — |
| Pten | −4.92 | 1.86 | 1.67 | 0.03 |
| Casp3 | −4.18 | 1.44 | −1.53 | −1.93 |
| Mapk1 | −4.09 | 2.94 | 1.88 | 1.89 |
| Lamb2 | −4.03 | — | — | — |
| Tgfa | −3.86 | −1.47 | 1.49 | — |
| Cdkn2b | −3.79 | — | — | — |
| Pik3r1 | −3.77 | 1.91 | — | — |
| Prkcb | −3.73 | 0.62 | −0.31 | −0.49 |
| Itgb1 | −3.18 | — | — | — |
| Ctnna1 | −2.95 | 1.63 | 2.1 | 1.96 |
| Birc2 | −2.85 | — | — | — |
| Crk | −2.78 | — | — | — |
| Kras | −2.58 | 1.86 | 1.61 | − |
| Smo | −2.45 | — | — | 1.23 |
| Met | −2.32 | — | 1.81 | 1.94 |
| Msh2 | −2.29 | — | — | — |
| Erbb2 | −2.24 | — | 1.64 | 1.73 |
| Cycs | −2.08 | 1.78 | — | — |
| Mapk3 | −1.98 | — | — | — |
| Tpr | −1.93 | 1.81 | 0.29 | 1.64 |
| Mtor | −1.75 | 1.33 | — | — |
| Cdc42 | −1.7 | −2.25 | −1.91 | −2.21 |
| Plcg2 | −1.63 | 1.98 | −1.41 | −1.46 |
| Tgfb1 | −1.54 | −2.17 | −1.37 | −1.44 |
| Mecom | 1.46 | 2.08 | — | 1.76 |
| Pdgfrb | 1.49 | — | — | 1.65 |
| Raf1 | 1.51 | −0.08 | −0.03 | −0.12 |
| Hdac1 | 1.52 | — | — | — |
| Tgfb3 | 1.83 | — | — | — |
| Axin2 | 2.12 | — | — | — |
| Gsk3b | 2.3 | — | 1.46 | 1.87 |
| Sos2 | 2.47 | −1.39 | — | — |

“—”: means that the gene expression is within the fold change range of −1.2 to 1.2.

The EMR data of AKI patients from the two clinical databases, MIMIC III and eICU, were analyzed. Drugs potentially effective in preventing AKI patients from death during ICU attendance were identified. Most of these drugs were already known as “life-saving” drugs. For example, metoprolol, a beta blocker, is used on heart failure patients and can prevent patients from death. Other drugs included in the drug set such as furosemide, magnesium sulfate, fluconazole, lisinopril, hydralazine, insulin, meropenem, and captopril had beneficial effects on accelerating the recovery of AKI patients. Therefore, the beneficial effect of other drugs with lack of literature support in the Cox result is also considerable.

It was noticed that several drugs showed “promoting” effects on patients' ICU death rate. All of these medications have already been suggested by literature reports, clinical trials, and/or guidelines to be potentially harmful or even forbidden in AKI therapy in ICU, such as morphine, norepinephrine, and fentanyl.

Among all the beneficial drugs, ondansetron was of most interest. Ondansetron is a selective antagonist on serotonin 5 HT-3 receptor, which is a receptor with wide distribution in human body. Last, ondansetron showed one of the best performances on mortality decreasing among all drugs. For the total 7,313 patients included in the study, the average death rate was 22.7%, however, for 1,171 patients who were using ondansetron, the average death rate dropped to 14.1%.

Such findings also were confirmed in AKI patients from the eICU database. In the eICU database, the indication bias was managed by comparing ondansetron with same effect drug (prochlorperazine). However, among 13,000 patients, there were only 314 patients who used prochlorperazine and only 55 patients who used prochlorperazine without using ondansetron at the same time. The limitation of sample size (55 patients) and overlap restricted the power of the analysis. As such, the PSM was conducted to adjust the baselines of the patients instead.

There is a chance that the lower mortality in ondansetron-treated group is the result of that using this antiemetic drug is a symbol of conscious, and rest ICU patients are likely to be unconscious. Thus, patients in ondansetron group may originally have had a milder disease. Therefore, the effect of ondansetron on AKI was evaluated to determine if the effect has some latent molecular mechanisms using gene expression signature. If there is a significant overlap of the genes regulated by the drug and disease, and the drug and disease are influencing the gene expression to opposite direction, the drug can be concluded to have therapeutic effect on this disease. The analysis revealed that there was an overlap of genes affected by both ondansetron and AKI. The most significant overlap happens on the genes down regulated by ondansetron and also upregulated by AKI.

Ondansetron is a serotonin 5-HT₃ receptor antagonist and 5-HT₃ receptor is a ligand-gated ion channel. Magnesium sulfate, a drug used for the treatment of acute nephritis in children also has voltage-gated calcium channel activity. It seems that ondansetron can hold the cells in an in-active state through inhibiting the ion flow, a key signaling transductor in the pathways involved in AKI progression.

The analysis was adjusted for other medications and comorbidies to mitigate the effects of co-founders in the Cox analysis, and PSM was used to alleviate the possible bias at the baseline in the validation step. However, the possibility of the co-founder effects, such as surgery, cannot be ruled out. From the gene expression data of molecular mechanism analysis, it was determined that ondansetron does have an effect on AKI.

### CONCLUSION

Through the analysis of EMR data from AKI patients in their ICU stays, drugs with beneficial effects by reduction in the ICU death rate were identified. Ondansetron was found to have beneficial effects on AKI recovery. This finding was further confirmed in the eICU database. The molecular mechanism of beneficial effects of ondansetron was also explored, which suggested that ondansetron can down-regulate AKI-related genes.

### Example 2

Additional combinations might strengthen the beneficial effects of ondansetron on AKI: A logistic regression analysis showed that other drugs can also decrease the in-ICU mortality of AKI patients. The data is summarized below in Table 8. These drugs can be potential candidates to be combined with ondansetron. The Z value is the regression coefficient divided by the standard error. If the Z value is too high in magnitude, the Z value indicates that the corresponding true regression coefficient is not 0 and the corresponding x-variable matters. Pr(>|Z|) is the p-value of significance of the Z value.

TABLE 8
Estimate coefficients and significance of drugs
that influence ICU mortality of patients with AKI

| Drug | Estimate | Std. Error | Z Value | Pr(>\|Z\|) |
|---|---|---|---|---|
| Warfarin | −1.485 | 0.177 | −8.41 | 4.15E−17 |
| Oxycodone | −1.070 | 0.137 | −7.83 | 4.96E−15 |
| Lisinopril | −0.857 | 0.200 | −4.28 | 1.89E−05 |
| Heparin | −0.651 | 0.104 | −6.29 | 3.11E−10 |
| Magnesium Sulfate | −0.560 | 0.101 | −5.56 | 2.77E−08 |
| Haloperidol | −0.553 | 0.125 | −4.44 | 9.18E−06 |
| Glucagon | −0.464 | 0.142 | −3.28 | 0.00104 |
| Metoprolol | −0.454 | 0.102 | −4.44 | 9.11E−06 |
| Furosemide | −0.359 | 0.101 | −3.55 | 0.000384 |
| Acetaminophen | −0.332 | 0.098 | −3.40 | 0.000663 |
| Hydralazine | −0.323 | 0.131 | −2.46 | 0.0137 |
| Hydromorphone | −0.299 | 0.132 | −2.26 | 0.0238 |

Z Value: Coefficient divided by standard error; Pr(>|Z|): significance that the corresponding coefficient is not 0.

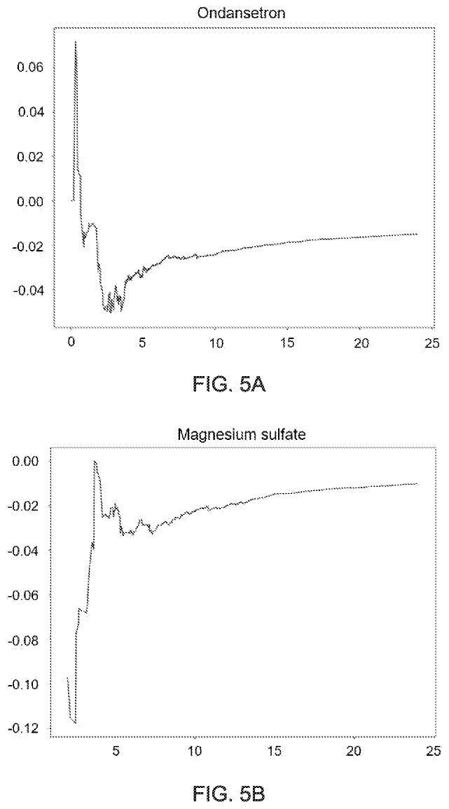

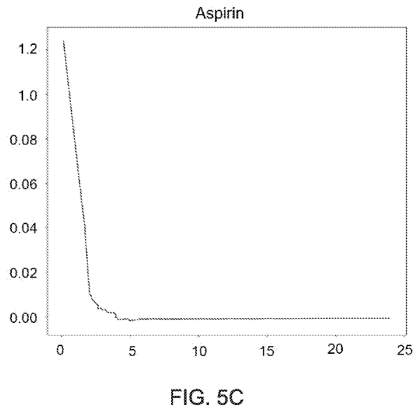

Timing of administration: AKI Patients in ICU usually are closely monitored on their physiological parameters such as creatinine level, a biomarker for AKI (AKI is diagnosed if serum creatinine increases by 0.3 milligrams per deciliter (mg/dl) (26.5 micromole per liter (μmol/l)) or more in 48 hours or rises to at least 1.5-fold from baseline within 7 days). FIGS. 5A-5C show that ondansetron can reach the maximum capability of decreasing creatinine level at a time of 2.5-4 hours after administration, while magnesium sulfate, a drug for acute nephritis, reaches maximum capability around 6-7 hours (the 2 hour valley is a fake one) after administration and aspirin has no effect on creatinine levels. The time of administration may be when the creatinine level reaches the threshold of AKI diagnosis.

Mode of administration: Considering that AKI patients in ICU usually are in critical condition, an intravenous (IV) injection or infusion was preferred.

Dosage: The most popular dosage for adult patients in ICU setting is 4 mg q8H (every 8 hours) and 3 times every day through IV injection. For treatment of adult AKI patients, the dosage can be higher but should not be more than 32 mg q8 h (from Drugbank “At the highest tested dose of 32 milligrams (mg), prolongation of the Fridericia-corrected QTc interval (QT/RRO.33=QTcF) was observed from 15 min to 4 hours after the start of the 15 minute infusion, with a maximum mean (upper limit of 90% confidence interval (CI)) difference in QTcF from placebo after baseline-correction of 19.6 (21.5) milliseconds (msec) at 20 minutes.”) and treatment could be as long as 7-10 days. For prevention, treatment may be 1-3 days.

Combinations: The following drugs may be combined with ondansetron because of data showing that the following drugs can also decrease the ICU mortality of AKI patients: warfarin; Lisinopril; heparin; magnesium sulfate; haloperidol; glucagon; metoprolol; furosemide; or hydralazine.

### Example 3—Anti-Emetic Usage and AKI Risk

Study Population: The study population excludes patients with end-stage kidney disease and excludes patients with Acute Kidney Injury on admission or prior to receiving drug.

Table 9 shows ondansetron, metoclopramide, and prochlorperazine usage for patients having AKI during their first encounter and patients having no AKI during their first encounter.

TABLE 9
Ondansetron, Metoclopramide, Prochlorperazine Usage

| | AKI During | No AKI During | |
|---|---|---|---|
| | First Encounter | First Encounter | |
| | (22,947) | (96,949) | p-value |
| Any MOP* usage prior to | 5,513 (24.02) | 42,863 (44.21) | <0.0001 |
| AKI (%) | | | |
| Ondansetron usage prior to | 5,231 (22.80) | 40,997 (42.29) | <0.0001 |
| AKI (%) | | | |
| Metoclopramide usage prior | 498 (2.17) | 5,001 (5.16) | <0.0001 |
| to AKI (%) | | | |
| Prochlorperazine usage prior | 352 (1.53) | 3,199 (3.30) | <0.0001 |
| to AKI (%) | | | |

*MOP = pooled use of any metoclopramide, ondansetron, or prochloroperazine.
Prior to AKI in patients without AKI = use of drug before discharge/death.

Table 10 shows anti-emetic drug use or no anti-emetic drug use in patients falling under various maximum KDIGO AKI categories.

TABLE 10
Max AKI with Pooled anti-emetic use

| MAX KDIGO AKI | No Anti-Emetic Use | Anti-Emetic Use |
|---|---|---|
| Category | (N = 67,422) | (N = 46,175) |
| 0 (%) | 40,365 (59.87) | 33,855 (73.32) |
| 1 (%) | 12,838 (19.04) | 6,441 (13.95) |
| 2 (%) | 6,531 (9.69) | 3,105 (6.72) |
| 3 (%) | 7,688 (11.40) | 2,774 (6.01) |

Anti-emetic use = use of drug before discharge/death or before AKI

Table 11 shows ondansetron use or no ondansetron use in patients falling under various maximum KDIGO AKI categories.

TABLE 11
Max AKI with Ondansetron Usage

| MAX KDIGO AKI | No Ondansetron Use | Ondansetron Use |
|---|---|---|
| Category | (N = 69,473) | (N = 44,124) |
| 0 (%) | 41,773 (60.13) | 32,447 (73.54) |
| 1 (%) | 13,136 (18.91) | 6,143 (13.92) |
| 2 (%) | 6,690 (9.63) | 2,946 (6.68) |
| 3 (%) | 7,874 (11.33) | 2,588 (5.87) |

Anti-emetic use = use of drug before discharge/death or before AKI

Table 12 shows metoclopramide use or no metoclopramide use in patients falling under various maximum KDIGO AKI categories. Metoclopramide is primarily a dopamine D₂ receptor antagonist anti-emetic.

TABLE 12
Max AKI with Metoclopramide Usage

| MAX KDIGO AKI | No Metoclopramide Use | | No Metoclopramide Use | |
|---|---|---|---|---|
| Category | (N = 108,317) | | (N =5,280) | |
| 0 (%) | 70,390 | (64.99) | 3,830 | (72.54) |
| 1 (%) | 18,537 | (17.11) | 742 | (14.05) |
| 2 (%) | 9,271 | (8.56) | 365 | (6.91) |
| 3 (%) | 10,119 | (9.34) | 343 | (6.50) |

Anti-emetic use = use of drug before discharge/death or before AKI

Table 13 shows prochlorperazine use or no prochlorperazine use in patients falling under various maximum KDIGO AKI categories. Prochlorperazine is primarily a dopamine receptor antagonist anti-emetic.

TABLE 13
Max AKI with Prochlorperazine Usage

| MAX KDIGO AKI | No Prochlorperazine Use | | No Prochlorperazine Use | |
|---|---|---|---|---|
| Category | (N = 110,211) | | (N = 3,386) | |
| 0 (%) | 71,732 | (65.09) | 2,488 | (73.48) |
| 1 (%) | 18,779 | (17.04) | 500 | (14.77) |
| 2 (%) | 9,437 | (8.56) | 199 | (5.88) |
| 3 (%) | 10,263 | (9.31) | 199 | (5.88) |

Anti-emetic use = use of drug before discharge/death or before AKI

Similar results for all 3 anti-emetics suggests indication bias.

Table 14 shows logistic regression results. This was a binary logistic regression with an outcome of experiencing any AKI within a first ICU encounter. The independent variable was use of anti-emetic prior to AKI (or before discharge if no AKI).

TABLE 14
Logistic Regression Results

| | Odds | p- | 95% CI | 95% CI |
|---|---|---|---|---|
| Medication | Ratio | value | lower | upper |
| MOP* | 0.399 | <0.001 | 0.386 | 0.412 |
| Ondansetron | 0.403 | <0.001 | 0.390 | 0.417 |
| Metoclopramide | 0.408 | <0.001 | 0.372 | 0.448 |
| Prochlorperazine | 0.457 | <0.001 | 0.409 | 0.510 |

*MOP = pooled use of any metoclopramide, ondansetron, or prochloroperazine.

Again, similar effects were seen for all three drugs.

Table 15 shows logistic regression results. This was a binary logistic regression with an outcome of experiencing AKI Category 2 or Category 3 vs. AKI Category 0 or Category 1. The independent variable was use of anti-emetic prior to AKI (or before discharge if no AKI).

TABLE 15
Logistic Regression Results

| | Odds | p- | 95% CI | 95% CI |
|---|---|---|---|---|
| Medication | Ratio | value | lower | upper |
| MOP* | 0.841 | <0.001 | 0.815 | 0.868 |
| Ondansetron | 0.824 | <0.001 | 0.798 | 0.851 |
| Metoclopramide | 1.033 | 0.79 | 0.953 | 1.120 |
| Prochlorperazine | 0.90 | 0.046 | 0.816 | 0.998 |

*MOP = pooled use of any metoclopramide, ondansetron, or prochloroperazine.

When only moderate or severe AKI (stages 2-3) were evaluated, only Ondansetron appeared to be significant.

Table 16 shows the average time to use each medication.

TABLE 16
Average time to use each medication

| | Average time | % of patients that received |
|---|---|---|
| Medication | to use (hours) | within average time |
| MOP* | 83 | 85% |
| Ondansetron | 85 | 85% |
| Metoclopramide | 160 | 63% |
| Prochlorperazine | 140 | 67% |

*MOP = pooled use of any metoclopramide, ondansetron, or prochloroperazine.

Table 17 shows logistic regression analysis. The outcome was experiencing any AKI. The dependent variable was the use of medication within the average time prior to AKI.

TABLE 17
Logistic Regression Results

| | Odds | p- | 95% CI | 95% CI |
|---|---|---|---|---|
| Medication | Ratio | value | lower | upper |
| MOP* | 0.927 | <0.001 | 0.901 | 0.954 |
| Ondansetron | 0.906 | <0.001 | 0.880 | 0.933 |
| Metoclopramide | 1.249 | <0.001 | 1.181 | 1.320 |
| Prochlorperazine | 0.988 | 0.750 | 0.920 | 1.062 |

*MOP = pooled use of any metoclopramide, ondansetron, or prochloroperazine.

When controlling for late outliers, only ondansetron appeared protective.

Table 18 shows anti-emetic use in mortality. The outcome was in-ICU death. The dependent variable was use of medication within average time prior to AKI.

TABLE 18
Anti-Emetic Use in Mortality

| | Odds | p- | 95% CI | 95% CI |
|---|---|---|---|---|
| Medication | Ratio | value | lower | upper |
| MOP* | 0.497 | <0.001 | 0.472 | 0.523 |
| Ondansetron | 0.460 | <0.001 | 0.437 | 0.485 |
| Metoclopramide | 1.270 | <0.001 | 1.163 | 1.387 |
| Prochlorperazine | 0.736 | <0.001 | 0.644 | 0.841 |

MOP = pooled use of any metoclopramide, ondansetron, or prochloroperazine.

Table 19 shows anti-emetic use in mortality with disease severity. The outcome was in-ICU death. The dependent variables were the use of medication within average time prior to AKI and Apache Score on ICU admission.

TABLE 19
Anti-Emetic Use in Mortality with Disease Severity

| | Odds | p- | 95% CI | 95% CI |
|---|---|---|---|---|
| Medication | Ratio | value | lower | upper |
| MOP* | 0.561 | <0.001 | 0.531 | 0.593 |
| Apache Score | 1.05 | <0.001 | 1.05 | 1.05 |
| Ondansetron | 0.528 | <0.001 | 0.500 | 0.559 |
| Apache Score | 1.05 | <0.001 | 1.05 | 1.05 |
| Metoclopramide | 1.08 | 0.111 | 0.982 | 1.191 |
| Apache Score | 1.05 | <0.001 | 1.05 | 1.05 |
| Prochlorperazine | 0.879 | 0.077 | 0.761 | 1.014 |
| Apache Score | 1.05 | <0.001 | 1.05 | 1.05 |

*MOP = pooled use of any metoclopramide, ondansetron, or prochloroperazine.

After controlling for disease severity, only Ondansetron was found to be beneficial.

### Example 4

Logistic regression was used to investigate associations between medications received and ICU mortality in patients with AKI in the MIMIC III database. Drugs associated with reduced mortality were then validated using the eICU database. Propensity score matching (PSM) was used for matching the patients' baseline severity of illness followed by a chi-square test to calculate the significance of drug use and mortality. Gene expression signatures were examined to explore the drug's molecular mechanism on AKI. While several drugs demonstrated potential beneficial effects on reducing mortality, most were used for potentially fatal illnesses (e.g., antibiotics, cardiac medications). Ondansetron was found to be correlated with lower mortality among AKI patients and was confirmed in a subsequent analysis using the eICU database.

### Materials and Methods

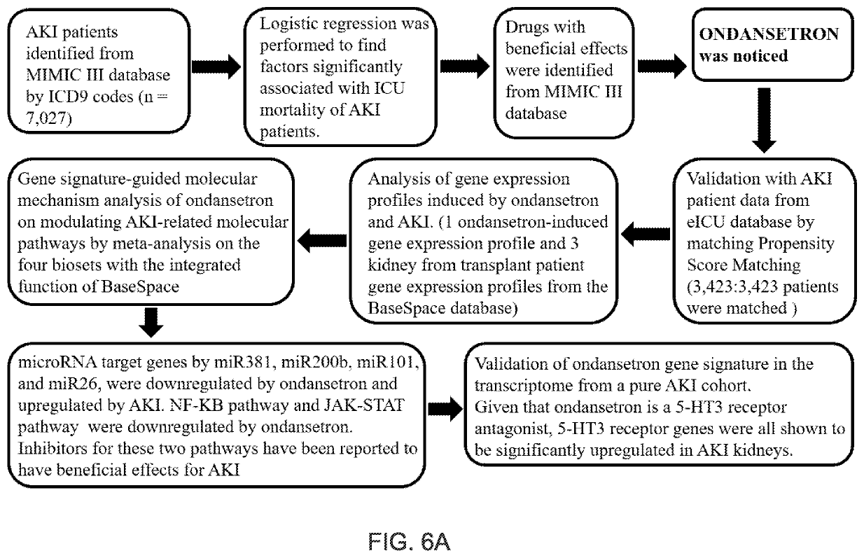

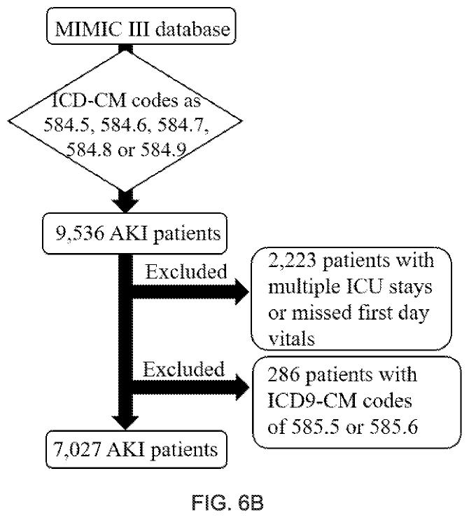

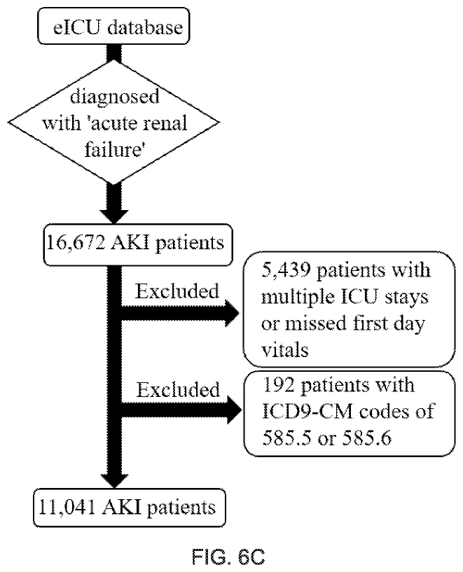

Research Design: FIGS. 6A, 6B, and 6C show the flow charts of the procedure for Example 4. The AKI patients were first identified from the MIMIC III database using International Classification of Diseases, Ninth Revision (ICD9) codes. The clinical data was extracted (age, gender, medication use information and other variables to determine patients' conditions). Logistic regression was used to select variables that showed significant beneficial effects on ICU mortality. Among these variables, drug(s) with potential beneficial effects were identified and literature searches were conducted to confirm the plausibility. Data from a second independent database (eICU) was used to validate our findings, and the focus was on drugs that were not expected to have a direct effect on survival from their primary use (e.g., drugs used to manage symptoms but not life-threatening conditions). Finally, gene signature analysis was used to find possible mechanisms for each drug candidate for their beneficial effects on AKI.

Data source: The study utilized data from MIMIC III v1.4, eICU, and Illumina BaseSpace software, as described above in Example 1.

Population selection criteria: The ICD9-CM codes 584.5, 584.6, 584.7, 584.8, and 584.9 were used to search the diagnosis table in the MIMIC III database to identify patients with AKI. For the eICU database, the keyword search “acute renal failure” was used in the diagnosis table to identify patients with AKI. Patients with multiple ICU stays or with first day vitals missing were excluded to simplify the calculation. Patients with ICD9-CM codes of 585.5 (Chronic kidney disease, Stage V) and 585.6 (End stage renal disease) were also excluded. Because the focus of this study was mortality in ICU stays for AKI patients, no censor strategies were applied. In other words, upon discharging from ICU units, the patients were either alive or dead.

Data extraction and missing values process: From the MIMIC III database, the demographic characteristics, physiological index, ICD9 codes, medications, laboratory tests, and vital status (alive or dead) upon ICU discharge were extracted. These variables were classified into three categories: first day vital information, medication use information, and other variables as described in Example 1. As Ondansetron is used for nausea and vomiting, especially in post-surgical patients, the surgical status of those patients were also considered. For MIMIC III database, the CPTEVENTS table on the SECTIONHEADER containing “Surgery” was searched. For the eICU database, the diagnosis table on the diagnosis string containing “surgery” was searched. Medication use information was filtered to be the top 50 most used drugs among these patients with AKI. From the eICU database, physiology characters, comorbidities situation, medications, and vital status on ICU discharge were extracted. The codes for comorbidities were from GitHub (see, https://github.com/MIT-LCP/mimic-code/blob/52d7df53348a6e25dfbe795c0e28c389efc40be9/mimic-iii/concepts/comorbidity/elixhauser_quan.sql. The codes for first day labs and vital information were also from GitHub (see, https://github.com/MIT-LCP/mimic-code/tree/main/mimic-iii/concepts/firstday). Missing values were found in physiological indexes such as average heart rate, average systolic blood pressure, average blood glucose and average albumin counts. For MIMIC III data, all variables containing missing values were continuous variables. The missing values were filled with means of the whole column (Brand, M. “Incremental singular value decomposition of uncertain data with missing values”, in European Conference on Computer Vision, 2002, Springer). The drug and disease induced DEGs were extracted using the Ilumina BaseSpace software.

Logistic regression to identify variables that significantly influence ICU mortality of patients with AKI: The extracted data was analyzed using multivariate logistic regression. Death within 24 hours of ICU discharge was considered as the primary outcome. Eleven variables were dropped because of collinearity between covariates for logistic regression, including paralysis, hypothyroidism, peptic ulcer disease, obesity, weight loss, blood loss anemia, deficiency anemias, drug abuse, psychoses, cardiac arrhythmia, and depression.

Validation of findings using the eICU database: The findings were validated with the eICU database. Patients' physiology characteristics (temperature, respiratory rate, heartrate, mean blood pressure and creatinine), surgical status, and their comorbidities situation (including comorbidities that affect larger than 5% patients in either treatment or non-treatment group) were used for propensity score matching (Rubin, D. B. et al. “Matching using estimated propensity scores: relating theory to practice”, Biometrics, 1996, 249-264) of the patient's baseline severity of illness followed by a chi-square test to calculate the significance of drug use and mortality. The Propensity score matching process was conducted using R package Match It function “matchit” (method=“nearest”, ratio=1, discard=“both”, caliper=0.05). The assumption that those matched patients will have similar physiology condition was made.

Investigation of possible molecular mechanisms by comparison of gene transcriptional profiles: The molecular mechanisms were investigated by comparison of gene transcriptional profiles, as described in Example 1. The drug-induced DEGs were used to search against disease-induced DEGs (three biosets, names can be found in Table 5) to find potential associations between these drugs and kidney diseases through the commonly modulated genes. The molecular pathways of those common genes involved were collected through meta-analysis function in BaseSpace to investigate possible molecular mechanisms for beneficial effects.

Validation of ondansetron gene signature in a human “pure AKI” cohort: To further elucidate the molecular mechanisms of ondansetron in AKI, the gene signature of ondansetron was validated in transcriptome data from a pure AKI cohort (GEO ID: GSE30718). Because some degree of AKI happens in all kidney transplantation patients, an excellent human AKI model can be found in early kidney transplants without rejection. In a prospective study of 234 kidney transplant biopsies for clinical indications, kidneys with rejection and kidney disease (other than AKI) by histologic criteria were excluded, and those with nondiagnostic suspicious histologic lesions were also excluded (Famulski, K. S., et al., “Molecular phenotypes of acute kidney injury in kidney transplants”, Journal of the American Society of Nephrology, 2012, 23(5): 948-958).

These criteria identified a “pure AKI” cohort of 28 biopsies with a mean age of 52 (16-75), 15 (57.6%) living donors and with mean eGFR of 26 ml/min (Famulski et al.). A total of 11 pristine protocol biopsies represented kidneys with a stable future function (at least 2 years of follow-up) after transplantation, no evidence for AKI or rejection by histology, and no clinical indication for biopsy (clinical or subclinical, before or after biopsy) were used as the controls in this study. The statistical comparison was obtained by estimated marginal means (also known as least-squares means) using R. Through orthology mapping, 1,333 gene expression alternations in the human AKI cohort were identified. The DEG were defined using a stringent threshold of 1.5-fold changes and a p-value of less than 0.0001.

Statistical Analysis: The logistic regression was performed in R using caret package to measure the association between patient death and variables. The x2 (chi-square) test was performed to evaluate the difference of death rates between target medication users and non-users. Matching process to balance the propensity score of the patient of both groups was conducted using R package Match It function “matchit” (method=“nearest”, ratio=1, discard=“none”, caliper=0.05). Student t-test was used to measure the mean difference between the two samples. All statistical t-tests were calculated by R. The Running Fisher algorithm is used by BaseSpace software to assess the statistical significance of overlapping between two gene sets, where p-values are computed by a Fisher's exact test. A p <0.05 was used as the threshold for statistical significance for all analyses except where stated otherwise.

Results Variables associated with ICU mortality in patients with AKI: From the MIMIC III database, 9,536 patients with AKI were identified, of whom 9,443 had completed information on demographics, ICU stay, and the first day vital information. Patients with multiple ICU stays were excluded to simplify the calculation, resulting in 7,313 unique patients. 286 patients with diagnosis codes of 585.5 or 585.6 were also excluded, leaving 7,027 AKI patients. The basic characteristic of patients in the MIMIC III cohort can be found in Table 20, where Level 1 is for patients with the disease and 0 is for patients without the disease listed. For gender, 0 is for female patients and 1 is for male patients.

TABLE 20
Basic characteristics of the MIMIC III patients

| | | Non- | | | | |
|---|---|---|---|---|---|---|
| | | ondansetron | | Ondansetron | | p- |
| | Level | group (N = 6,142) | | group (N = 1,171) | | value |
| Heart rate (mean (SD)) | | 87.14 | (17.14) | 88.48 | (17.57) | 0.015 |
| Systolic blood pressure (mean | | 115.85 | (17.64) | 118.28 | (18.15) | <0.001 |
| (SD)) | | | | | | |
| Diastolic blood pressure (mean | | 58.52 | (11.12) | 60.97 | (11.93) | <0.001 |
| (SD)) | | | | | | |
| Mean blood pressure (mean (SD)) | | 75.26 | (11.43) | 76.71 | (12.14) | <0.001 |
| Respiratory (mean (SD)) | | 20.15 | (4.43) | 19.59 | (4.14) | <0.001 |
| Temperature (mean (SD)) | | 36.72 | (0.73) | 36.76 | (0.67) | 0.08 |
| oxygen saturation (mean (SD)) | | 96.60 | (3.43) | 96.78 | (2.59) | 0.081 |
| Glucose (mean (SD)) | | 147.39 | (52.77) | 144.80 | (49.19) | 0.12 |
| Congestive heart failure (%) | 0 | 6125 | (99.7) | 1166 | (99.6) | 0.569 |
| | 1 | 17 | (0.3) | 5 | (0.4) | |
| Pulmonary circulation (%) | 0 | 6094 | (99.2) | 1159 | (99.0) | 0.504 |
| | 1 | 48 | (0.8) | 12 | (1.0) | |
| Peripheral vascular (%) | 0 | 5972 | (97.2) | 1149 | (98.1) | 0.1 |
| | 1 | 170 | (2.8) | 22 | (1.9) | |
| Hypertension (%) | 0 | 6140 | (100.0) | 1170 | (99.9) | 0.975 |
| | 1 | 2 | (0.0) | 1 | (0.1) | |
| Age (mean (SD)) | | 88.53 | (68.73) | 74.47 | (53.16) | <0.001 |
| Anion gap (mean (SD)) | | 16.16 | (4.25) | 15.99 | (4.03) | 0.201 |
| Albumin (mean (SD)) | | 3.02 | (0.50) | 3.07 | (0.51) | <0.001 |
| Bicarbonate (mean (SD)) | | 22.01 | (5.10) | 21.80 | (4.84) | 0.18 |
| Bilirubin (mean (SD)) | | 2.46 | (4.58) | 2.50 | (4.88) | 0.789 |
| Creatinine (mean (SD)) | | 2.13 | (1.69) | 2.22 | (1.99) | 0.125 |
| Glucose (mean (SD)) | | 152.96 | (67.01) | 149.56 | (64.42) | 0.109 |
| Hematocrit (mean (SD)) | | 31.97 | (5.59) | 31.54 | (5.63) | 0.016 |
| Hemoglobin (mean (SD)) | | 10.67 | (1.93) | 10.53 | (1.97) | 0.024 |
| Lactate (mean (SD)) | | 2.87 | (2.12) | 2.60 | (1.59) | <0.001 |
| Platelet (mean (SD)) | | 216.30 | (120.48) | 219.35 | (120.68) | 0.426 |
| Potassium (mean (SD)) | | 4.34 | (0.66) | 4.29 | (0.68) | 0.023 |
| Sodium (mean (SD)) | | 138.56 | (5.80) | 137.61 | (4.82) | <0.001 |
| Blood urea nitrogen (mean (SD)) | | 43.20 | (27.71) | 40.40 | (28.36) | 0.002 |
| White blood cell (mean (SD)) | | 13.19 | (11.07) | 12.73 | (9.94) | 0.19 |
| Stroke (%) | 0 | 5864 | (95.5) | 1124 | (96.0) | 0.482 |
| | 1 | 278 | (4.5) | 47 | (4.0) | |
| GENDER (%) | 0 | 2597 | (42.3) | 569 | (48.6) | <0.001 |
| | 1 | 3545 | (57.7) | 602 | (51.4) | |

A Chi-Squared Test was used for categorical variables and a t-test was used for continuous variables. The information from these 7,027 patients was used to build a logistic regression model. Factors that contributed significantly to the prediction of ICU mortality (95% CI) were identified. Table 21 lists variables with p-values of <0.05. The accuracy, and AUC (area under curve) of this logistic regression model were 0.838 and 0.843, respectively. Estimate coefficients show two sides of the effect, positive and negative. A negative coefficient means that a factor is associated with increased survival vice versa. Death rates among patients with AKI who had ever used the top 50 drugs can be found in Table 22.

TABLE 21
Estimated coefficients and significance of factors
that influence ICU mortality of patients with AKI

| | | 95% Confidence interval | | |
|---|---|---|---|---|
| Variables | Coefficient | (upper and lower limits) | | Pr(>\|Z\|) |
| Warfarin | −0.115 | −0.139 | −0.090 | 2.971E−20 |
| Oxycodone | −0.089 | −0.110 | −0.068 | 2.456E−16 |
| Haloperidol | −0.063 | −0.086 | −0.039 | 2.224E−07 |
| Magnesium Sulfate | −0.054 | −0.071 | −0.037 | 8.970E−10 |
| Heparin | −0.053 | −0.071 | −0.036 | 3.328E−09 |
| Metoprolol | −0.051 | −0.070 | −0.033 | 3.653E−08 |
| Glucagon | −0.050 | −0.074 | −0.026 | 4.959E−05 |
| Lisinopril | −0.049 | −0.075 | −0.024 | 1.323E−04 |
| Captopril | −0.044 | −0.076 | −0.011 | 0.008 |
| Acetaminophen | −0.040 | −0.058 | −0.023 | 5.134E−06 |
| Furosemide | −0.036 | −0.054 | −0.018 | 8.060E−05 |
| Ondansetron | −0.035 | −0.056 | −0.014 | 0.001 |
| Hydralazine | −0.035 | −0.058 | −0.011 | 0.004 |
| Docusate | −0.028 | −0.050 | −0.006 | 0.012 |
| Pantoprazole | −0.025 | −0.042 | −0.008 | 0.004 |
| Hydromorphone | −0.024 | −0.048 | −0.001 | 0.042 |
| Bisacodyl | −0.021 | −0.041 | −0.001 | 0.043 |
| Creatinine average | −0.020 | −0.027 | −0.013 | 5.915E−08 |
| SpO2 mean | −0.006 | −0.009 | −0.004 | 8.278E−09 |
| Platelet average | −1.165E−04 | −1.870E−04 | −4.603E−05 | 0.001 |
| BUN average | 4.633E−04 | 5.097E−05 | 0.001 | 0.028 |
| PTT average | 0.001 | 0.001 | 0.002 | 3.457E−13 |
| Bilirubin average | 0.002 | 0.000 | 0.004 | 0.037 |
| Resprate mean | 0.005 | 0.003 | 0.007 | 1.840E−07 |
| Sapsii | 0.006 | 0.005 | 0.007 | 1.184E−44 |
| Anion gap average | 0.011 | 0.004 | 0.018 | 0.003 |
| Lactate average | 0.016 | 0.011 | 0.021 | 1.400E−09 |
| Potassium average | 0.020 | 0.006 | 0.035 | 0.007 |
| Midazolam | 0.027 | 0.001 | 0.054 | 0.043 |
| Lorazepam | 0.031 | 0.014 | 0.049 | 0.001 |
| Stroke | 0.066 | 0.029 | 0.103 | 4.373E−04 |
| Fentanyl | 0.076 | 0.050 | 0.102 | 8.943E−09 |
| Meropenem | 0.076 | 0.047 | 0.106 | 4.689E−07 |
| Solid tumor | 0.078 | 0.017 | 0.140 | 0.012 |
| Amiodarone | 0.090 | 0.065 | 0.115 | 2.719E−12 |
| Norepinephrine | 0.167 | 0.144 | 0.190 | 6.629E−46 |
| Morphine | 0.198 | 0.181 | 0.215 | 7.624E−109 |
| Coagulopathy | 0.809 | 0.186 | 1.431 | 0.011 |

PTT: Partial thromboplastin time; SpO2: Oxygen saturation; Bun: blood urea nitrogen.

TABLE 22
Detailed ICU death rates among 7,313 patients with AKI with top
50 used drugs in MIMIC III database and 12,676 patients with AKI
with top 50 used drugs in eICU database (NA: not available)

| | Death rate in | Death rate in |
|---|---|---|
| | MIMIC III database | eICU database |
| Drug name | (per 1,000 patients) | (per 1,000 patients) |
| Acetaminophen | 181.26 | 123.47 |
| Albuterol | 257.20 | 174.59 |
| Amiodarone | 338.95 | 269.89 |
| Aspirin | 178.97 | 126.32 |
| Atorvastatin | 156.06 | 88.66 |
| Bisacodyl | 194.46 | 156.59 |
| Captopril | 133.04 | NA |
| Cefepime | 340.81 | 228.51 |
| Chlorhexidine | 328.48 | 222.98 |
| Dexamethasone | 375 | 58.14 |
| Diltiazem | 274.96 | 121.74 |
| Docusate | 178.57 | 136.48 |
| Famotidine | 260.79 | 184.42 |
| Fentanyl | 377.87 | 239.60 |
| Fluconazole | 333.33 | NA |
| Furosemide | 214.64 | 120.87 |
| Glucagon | 176.04 | 129.74 |
| Haloperidol | 194.85 | 201.23 |
| Heparin | 210.41 | 143.03 |
| Hydralazine | 155.12 | 80.60 |
| Hydromorphone | 166.18 | 141.20 |
| Insulin | 241.12 | 172.01 |
| Ipratropium | 258.87 | 172.83 |
| Lactulose | 326.35 | 233.56 |
| Levofloxacin | 270.20 | 146.84 |
| Levothyroxine | 218.39 | 109.02 |
| Lisinopril | 53.11 | 21.71 |
| Lorazepam | 285.59 | 228.92 |
| Magnesium Sulfate | 203.36 | 172.14 |
| Meropenem | 460.50 | 269.96 |
| Metoclopramide | 248.67 | 158.32 |
| Metoprolol | 169.99 | 107.87 |
| Metronidazole | 338.05 | 230.12 |
| Midazolam | 899.95 | 273.07 |
| Morphine | 376.48 | 234.81 |
| Nitroglycerin | 192.42 | 758.55 |
| Norepinephrine | 497.51 | 336.62 |
| Ondansetron | 140.91 | 124.87 |
| Oxycodone | 94.98 | 55.56 |
| Pantoprazole | 254.46 | 151.82 |
| Phytonadione | 364.17 | 335.33 |
| Piperacillin | 336.96 | 200.39 |
| Prednisone | 191.30 | 60.49 |
| Propofol | 287.82 | 261.45 |
| Senna | NA | 200 |
| Tacrolimus | 153.28 | 40 |
| Vancomycin | 300.03 | 229.29 |
| Warfarin | 67.31 | 35.34 |
| All | 227.13 | 152.67 |

Drugs with potentially beneficial effects on AKI mortality: A literature search was conducted on all drugs identified to have effects of mortality (see, Table 2 in Example 1). Nine of 22 drugs with negative coefficients in Table 21 were reported to have a beneficial effect on decreasing ICU mortality. Among all drugs, ondansetron stood out as an anti-emetic, which seemed to be less related on preventing death and AKI recovery. Ondansetron showed the best performance on decreasing ICU mortality in patients with AKI with the least connection of indication on a previously identified life-saving effect.

Validation of beneficial effects of ondansetron using the eICU database: 14,338 patients with AKI from the eICU database were identified. 5,439 patients were excluded because of missing data or having multiple ICU stays. 192 patients with ICD9-CM codes of 585.5 or 585.6 were also excluded. 11,041 AKI patients from eICU database were obtained. The mean baseline characters values of patients' receiving/not receiving ondansetron were significantly different. A 1:1 propensity score matching (3,423:3,423 patients were matched) to balance the patients' physiology condition between treatment and control group. Detailed basic characteristics of eICU patients before and after matching can be found in Tables 23 and 24, respectively. Level 1 is for patients with the disease and 0 is for patients without the disease listed.

TABLE 23
The detailed basic characteristics of eICU patients before matching

| | | Non- | | Ondansetron | | |
|---|---|---|---|---|---|---|
| | | ondansetron | | users | | p- |
| | Level | users (N = 7,626) | | (N = 3,446) | | value |
| Gender (%) | Female | 3317 | (43.5) | 1534 | (44.5) | 0.486 |
| | Male | 4308 | (56.5) | 1912 | (55.5) | |
| | Unknown | 1 | (0.0) | 0 | (0.0) | |
| Age (%) | <=49 | 1070 | (14.0) | 565 | (16.4) | <0.001 |
| | >49 | 1286 | (16.9) | 657 | (19.1) | |
| | >59 | 1736 | (22.8) | 768 | (22.3) | |
| | >69 | 1736 | (22.8) | 790 | (22.9) | |
| | >79 | 1442 | (18.9) | 532 | (15.4) | |
| | >89 | 356 | (4.7) | 134 | (3.9) | |
| Congestive heart failure (%) | 0 | 6390 | (83.8) | 2979 | (86.4) | <0.001 |
| | 1 | 1236 | (16.2) | 467 | (13.6) | |
| Cardiac arrhythmias (%) | 0 | 5741 | (75.3) | 2647 | (76.8) | 0.086 |
| | 1 | 1885 | (24.7) | 799 | (23.2) | |
| Hypertension (%) | 0 | 6228 | (81.7) | 2834 | (82.2) | 0.486 |
| | 1 | 1398 | (18.3) | 612 | (17.8) | |
| Hypothyroidism (%) | 0 | 7243 | (95.0) | 3319 | (96.3) | 0.002 |
| | 1 | 383 | (5.0) | 127 | (3.7) | |
| Coagulopathy (%) | 0 | 7057 | (92.5) | 3237 | (93.9) | 0.009 |
| | 1 | 569 | (7.5) | 209 | (6.1) | |
| Electrolyte | 0 | 5622 | (73.7) | 2594 | (75.3) | 0.088 |
| disorder (%) | 1 | 2004 | (26.3) | 852 | (24.7) | |
| Diabetes (%) | 0 | 6020 | (78.9) | 2851 | (82.7) | <0.001 |
| | 1 | 1606 | (21.1) | 595 | (17.3) | |
| Liver disease (%) | 0 | 6889 | (90.3) | 3127 | (90.7) | 0.522 |
| | 1 | 737 | (9.7) | 319 | (9.3) | |
| COPD (%) | 0 | 6803 | (89.2) | 3168 | (91.9) | <0.001 |
| | 1 | 823 | (10.8) | 278 | (8.1) | |
| Tumor (%) | 0 | 7250 | (95.1) | 3239 | (94.0) | 0.021 |
| | 1 | 376 | (4.9) | 207 | (6.0) | |
| Respiratory failure (%) | 0 | 4301 | (56.4) | 2189 | (63.5) | <0.001 |
| | 1 | 3325 | (43.6) | 1257 | (36.5) | |
| Temperature (mean (SD)) | | 36.28 | (1.29) | 36.31 | (1.17) | 0.247 |
| Respiratory rate (mean (SD)) | | 26.60 | (14.27) | 27.72 | (15.13) | <0.001 |
| Heart rate (mean (SD)) | | 105.76 | (31.40) | 105.56 | (31.18) | 0.75 |
| Mean blood pressure (mean | | 81.74 | (45.20) | 82.57 | (45.23) | 0.371 |
| (SD)) | | | | | | |
| Creatinine (mean (SD)) | | 2.79 | (2.27) | 2.94 | (2.55) | 0.002 |

TABLE 24
The detailed basic characteristics of eICU patients before matching

| | | Non- | | | | |
|---|---|---|---|---|---|---|
| | | ondansetron | | Ondansetron | | |
| | | users | | users | | p- |
| | level | (N = 7,626) | | (N = 3,446) | | value |
| Gender (%) | Female | 3317 | (43.5) | 1534 | (44.5) | 0.486 |
| | Male | 4308 | (56.5) | 1912 | (55.5) | |
| | Unknown | 1 | (0.0) | 0 | (0.0) | |
| Age (%) | <=49 | 1070 | (14.0) | 565 | (16.4) | <0.001 |
| | >49 | 1286 | (16.9) | 657 | (19.1) | |
| | >59 | 1736 | (22.8) | 768 | (22.3) | |
| | >69 | 1736 | (22.8) | 790 | (22.9) | |
| | >79 | 1442 | (18.9) | 532 | (15.4) | |
| | >89 | 356 | (4.7) | 134 | (3.9) | |
| Congestive heart failure (%) | 0 | 6390 | (83.8) | 2979 | (86.4) | <0.001 |
| | 1 | 1236 | (16.2) | 467 | (13.6) | |
| Cardiac arrhythmias (%) | 0 | 5741 | (75.3) | 2647 | (76.8) | 0.086 |
| | 1 | 1885 | (24.7) | 799 | (23.2) | |
| Hypertension (%) | 0 | 6228 | (81.7) | 2834 | (82.2) | 0.486 |
| | 1 | 1398 | (18.3) | 612 | (17.8) | |
| Hypothyroidism (%) | 0 | 7243 | (95.0) | 3319 | (96.3) | 0.002 |
| | 1 | 383 | (5.0) | 127 | (3.7) | |
| Coagulopathy (%) | 0 | 7057 | (92.5) | 3237 | (93.9) | 0.009 |
| | 1 | 569 | (7.5) | 209 | (6.1) | |
| Electrolyte | 0 | 5622 | (73.7) | 2594 | (75.3) | 0.088 |
| disorder (%) | 1 | 2004 | (26.3) | 852 | (24.7) | |
| Diabetes (%) | 0 | 6020 | (78.9) | 2851 | (82.7) | <0.001 |
| | 1 | 1606 | (21.1) | 595 | (17.3) | |
| Liver disease (%) | 0 | 6889 | (90.3) | 3127 | (90.7) | 0.522 |
| | 1 | 737 | (9.7) | 319 | (9.3) | |
| COPD (%) | 0 | 6803 | (89.2) | 3168 | (91.9) | <0.001 |
| | 1 | 823 | (10.8) | 278 | (8.1) | |
| Tumor (%) | 0 | 7250 | (95.1) | 3239 | (94.0) | 0.021 |
| | 1 | 376 | (4.9) | 207 | (6.0) | |
| Respiratory failure (%) | 0 | 4301 | (56.4) | 2189 | (63.5) | <0.001 |
| | 1 | 3325 | (43.6) | 1257 | (36.5) | |
| Temperature (mean (SD)) | | 36.28 | (1.29) | 36.31 | (1.17) | 0.247 |
| Respiratory rate (mean (SD)) | | 26.60 | (14.27) | 27.72 | (15.13) | <0.001 |
| Heart rate (mean (SD)) | | 105.76 | (31.40) | 105.56 | (31.18) | 0.75 |
| Mean blood pressure (mean (SD)) | | 81.74 | (45.20) | 82.57 | (45.23) | 0.371 |
| Creatinine (mean (SD)) | | 2.79 | (2.27) | 2.94 | (2.55) | 0.002 |

After the baseline adjustment by propensity score matching, a significantly lower ICU mortality (12.56%) in the ondansetron group than in the non-ondansetron matched control group (15.16%), P=0.002085 was observed (Table 25). Death rates among patients with AKI with the 50 most frequently used drugs from MIMIC III and eICU can be found in Table 22.

TABLE 25
The contingency table of the death events occurred
in patients receiving/not receiving ondansetron
(p-value is calculated with chi-square test)

| | Non-Ondansetron | Ondansetron | Total | p-value |
|---|---|---|---|---|
| Death | 519 | 430 | 949 | 0.002085 |
| Alive | 2904 | 2993 | 5897 | |
| Total | 3423 | 3423 | 6846 | |

Molecular mechanism study by gene expression signatures: The gene expression profiles induced by ondansetron and AKI were further analyzed. As described above in Example 1, through the BaseSpace database, one ondansetron dataset from the Chemical Effects in Biological Systems database was located (Waters) where intestines from rats were treated with ondansetron in vivo and assayed for expression. The “intestine of rats+ondansetron at 84 mg-kg in water by oral gavage 0.25d vs. vehicle” bioset was used to mimic the acute effects of this drug. A detailed comparison is shown in FIG. 4 and the Biosets are provided in Table 5. Ondansetron and AKI (“Kidney from transplant patient with toxic drug effects and UTI_vs_normal kidney” (Bioset 2)) have 1,815 and 3,289 differentially expressed genes (DEGs), respectively, and they shared 269 common DEGs with significant p-values of 4.0E-8. Among those overlapping DEGs, 61 were both upregulated and 53 were both downregulated. A total of 55 genes were upregulated by ondansetron but were downregulated by AKI, while 116 genes were downregulated by ondansetron but were upregulated by AKI in Bioset 2.

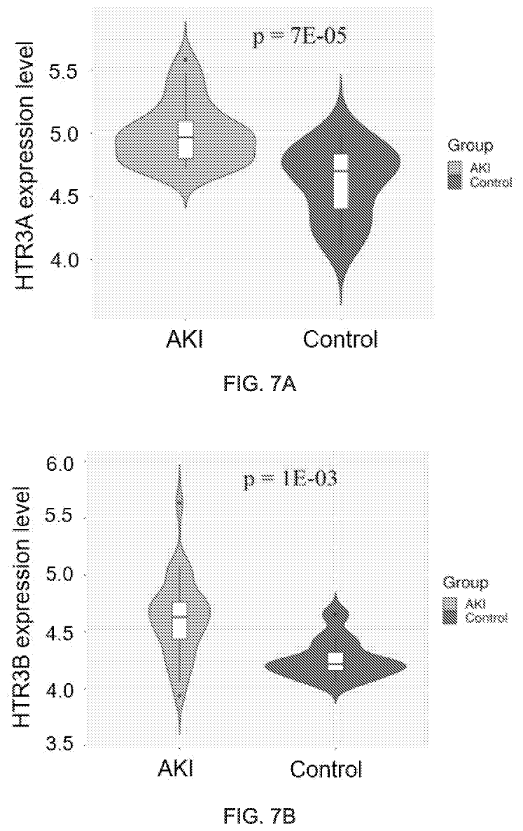

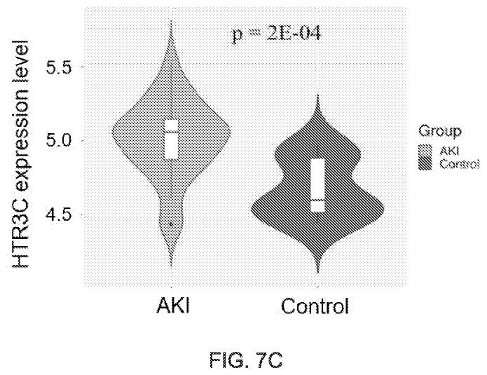

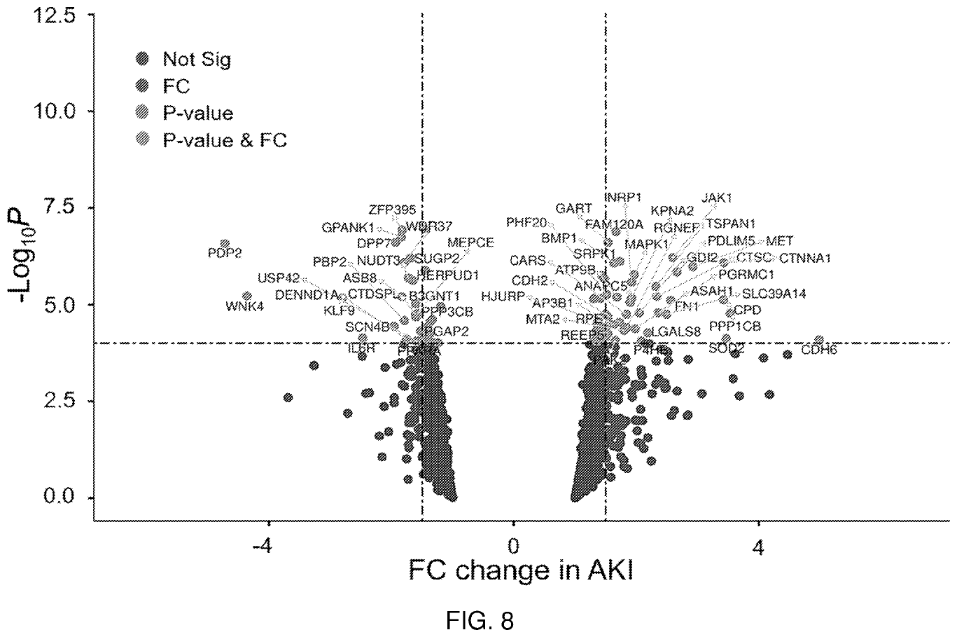

Validation of ondansetron gene signature in the transcriptome from a pure AKI cohort: Given that ondansetron is a 5-HT₃ receptor antagonist, the transcriptomes of 5-HT₃ receptor genes (HTR3A, HTR3B and HTR3C) were analyzed. By neutralizing the ubiquitous minor changes inevitably induced by the kidney transplant process, the comparison of AKI kidneys to histologically pristine protocol biopsies of stable transplants will reveal the molecular features of AKI. In this transcriptome study, 5-HT₃ receptor genes were all shown to be significantly upregulated (FIGS. 7A-7C). A volcano plot of the comparison results between the AKI and pristine protocol biopsy demonstrated significant positive and negative gene changes among the Ondansetron bioset. The volcano plot showed that JAK1, MAPK1, CTNNA1 and MET were upregulated in AKI by both fold change (FC) and P-values (FIG. 8). In FIG. 8, the area of interest is the top right of the volcano plot, where the fold change (FC) is greater than a certain threshold (indicated by the dashed line) and the negative logarithm base 10 (−log₁₀) of the p-value is greater than a different threshold (indicated by the dashed line). Of note, Rela is not in the upregulated gene list because the fold change was 1.49, which is around the threshold. Conversely, FN1, which displayed little change in the other three biosets above, changed significantly. FN1 was reported to be associated with AKI by the comparative toxicogenomics database (Davis, A. P., et al., “The Comparative Toxicogenomics Database: update 2019”, Nucleic Acids Research, 2018, 47(D1):D948-D954) with 499 references. Among the top 30 genes that were inversely correlated with estimated glomerular filtration rate (eGFR) at the time of biopsy in AKI biopsies, 8 genes were transcriptionally modulated by ondansetron. All 8 genes were upregulated in the human AKI cohort, and interestedly, 6 out of 8 genes whose gene expression was downregulated by Ondansetron were observed (Table 26). The genome-wide expression changes of all ondansetron pharmacological signature genes in this cohort were examined. The number of differentially expressed genes (AKI versus control) was significantly enriched with a p-value of 2.2E-11.

TABLE 26
Genes whose expressions were negatively correlated
with eGFR were also modulated by Ondansetron.

| | | Correlation of | Gene expression mRNA | | Gene |
|---|---|---|---|---|---|
| | | gene mRNA | change in GSE30718 | | expression |
| | | expression | (AKI vs. normal | | fold |
| | | with eGFR in | control) | | change by |
| Gene | | human AKI | | Fold | Ondansetron |
| Symbol | Gene title | cohort | p-value | change | treatment |
| KPNA2 | Karyopherin a2 | −0.72 | 6.73E−06 | 1.9 | 1.68 |
| | (RAG cohort 1, | | | | |
| | importin a1) | | | | |
| CASP1 | Caspase 1, | −0.72 | 1.00E−03 | 2.1 | −8.03 |
| | apoptosis-related | | | | |
| | cysteine | | | | |
| | peptidase (IL-1, b, | | | | |
| | convertase) | | | | |
| TFPI | Tissue factor | −0.7 | 0.01 | 1.5 | 4.25 |
| | pathway inhibitor | | | | |
| | (lipoprotein | | | | |
| | associated | | | | |
| | coagulation | | | | |
| | inhibitor) | | | | |
| AMACR | a-methylacyl-CoA | −0.68 | 2.80E−04 | 2.3 | −2.38 |
| | racemase | | | | |
| GBP2 | Guanylate binding | −0.67 | 1.00E−03 | 2.1 | −4.71 |
| | protein 2, IFN- | | | | |
| | inducible | | | | |
| MCL1 | Myeloid cell | −0.66 | 3.90E−05 | 2.8 | −3.07 |
| | leukemia sequence | | | | |
| | 1 (BCL2-related) | | | | |
| MET | Met proto- | −0.66 | 1.05E−06 | 2.9 | −2.32 |
| | oncogene | | | | |
| | (hepatocyte growth | | | | |
| | factor receptor) | | | | |
| CPD | Carboxypeptidase | −0.66 | 7.50E−06 | 3.4 | −2.4 |
| | D | | | | |

Among the drugs that were found to be associated with reduced mortality in critically ill patients with AKI, ondansetron was of the most interest. It is an anti-emetic drug and is a selective antagonist on the serotonin (5-HT₃) receptor (Wilde, M. I. et al. “Ondansetron”, Drugs, 1996, 52(5): 773-794), which is a receptor with wide distribution in the human body (Derkach, V. et al. “5-HT₃ receptors are membrane ion channels”, Nature, 1989, 339(6227):706-709) and its expression is upregulated in AKI (FIG. 8).

As discussed above in Example 1, the lower mortality in the ondansetron-treated group might be due to indication bias. Ondansetron is indicated for nausea which only awake, communicative patients can report. Patients in the ondansetron group may have had lower disease severity. This possibility was controlled for by matching propensity score of ondansetron users with non-users and a significant, albeit smaller, impact on mortality in this matched analysis was found.

In the analysis of eICU patients, the Number Need to Treat (NNT) was 38.46 (95% confidence interval 23.61, 103.72). This relatively high NNT may be due to the fact that patients were not prescribed ondansetron for treatment of AKI but for prevention/treatment of nausea and vomiting. These patients received comparably little dose in an irregular way. For example, when in the eICU database, there were 6,685 records of ondansetron use for 3,848 patients. Among these records, 5,849 (87.5%) were labeled as “prn” (as needed). For route of administration, 692 records were oral and 5,729 records were labeled as injections.

The possible molecular mechanisms were also explored by comparing gene expression signatures. If the genes regulated by the drug and the disease are in the opposite direction but show a significant overlap, the drug may have a potential therapeutic effect on the disease. The analysis revealed an overlap of genes affected by both ondansetron and AKI. The most significant overlap occurred on genes that are upregulated by AKI but downregulated by ondansetron. Furthermore, by validating the ondansetron gene signature in the “pure AKI” cohort, 5-HT₃ receptor genes were significantly upregulated in patients with AKI. The potential beneficial effect of ondansetron on AKI has support from molecular mechanisms.

To investigate the possible molecular mechanism of ondansetron on modulating AKI-related molecular pathways, a meta-analysis on four biosets with the integrated function of BaseSpace was performed. As discussed above in Example 1, pathways in cancer, microRNA target genes by miR381, miR200b, miR101, and miR26, were downregulated (see, Table 6 of Example 1). Furthermore, miR381 was reported to play a role in rat models of renal ischemia reperfusion injury (Zheng, G. H., et al., “MicroRNA-381-induced down-regulation of CXCR4 promotes the proliferation of renal tubular epithelial cells in rat models of renal ischemia reperfusion injury”, Journal of Cellular Biochemistry, 2018, 119(4):3149-3161). Target genes miR200b, miR101, and miR26 can be used as biomarkers for AKI (Kito et al., Aguado-Fraile et al.). As shown in Table 7 of Example 1, among the downregulated genes, Rela and Jak1 are the key proteins in the NF-κB pathway and JAK-STAT pathway, respectively. In comparison, these two genes are upregulated in AKI. Inhibitors for these two pathways have been reported to have beneficial effects for AKI (Si et al.; Ozkok et al.).

The analysis was adjusted for other medications and comorbidities to mitigate the effects of confounders in logistic regression, and matching was used to help alleviate the possible bias in baseline severity of illness in the validation step. However, the possibility of unknown confounder effects cannot be ruled out. From gene expression data of molecular mechanism analysis, ondansetron was shown to have an effect on pathways relevant to AKI.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. A method for treatment of acute kidney injury in a patient in need thereof, comprising administering to the patient a 5-HT₃-targeting drug, a 5-HT_{3A}-targeting drug, or a pharmaceutically-acceptable salt or solvate of a 5-HT₃-targeting drug or a 5-HT_{3A}-targeting drug, in an amount effective to treat acute kidney injury in the patient.

2. The method of claim 1 for treatment of a critically ill patient, comprising administering to the patient a 5-HT₃-targeting drug, a 5-HT_{3A}-targeting drug, or a pharmaceutically-acceptable salt or solvate of a 5-HT₃-targeting drug or a 5-HT_{3A}-targeting drug, in an amount effective to reduce risk of acute kidney injury in the patient.

3. The method of claim 2, wherein the critically ill patient is: an intensive care unit patient, a critical care unit patient, a patient requiring intensive care or critical care, a sepsis patient, a patient recovering from major surgery (a surgery in which a body cavity is entered), a patient exposed to a nephrotoxin, a patient having a microbial infection that increases risk of acute kidney injury, a kidney transplant recipient, a burn patient, or a critically-injured patient.

4. The method of claim 1, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is a 5-HT₃ antagonist, a 5-HT_{3A} antagonist, or a pharmaceutically-acceptable salt or solvate of a 5-HT₃ antagonist or a 5-HT_{3A} antagonist.

5. The method of claim 1, wherein 5-HT₃-targeting drug, the 5-HT_{3A}-targeting drug, or the pharmaceutically-acceptable salt or solvate of a 5-HT₃-targeting drug or a 5-HT_{3A}-targeting drug is one or more of tubocurarine, clozapine, aripiprazole, olanzapine, and metoclopramide, or a pharmaceutically-acceptable salt or solvate of any of the preceding.

6. The method of claim 1, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is one or more of ondansetron, granisetron, dolasetron, palonosetron, alosetron, cilansetron, tropisetron, ramosetron, or a pharmaceutically-acceptable salt or solvate of any of the preceding.

7. The method of claim 1, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is one or more of ondansetron, granisetron, dolasetron, palonosetron, alosetron, cilansetron, tropisetron, ramosetron, or a pharmaceutically-acceptable salt or solvate of any of the preceding.

8. The method of claim 1, wherein the patient is in an intensive care unit.

9. The method of claim 1, wherein the patient has a viral infection.

10. The method of claim 1, wherein the patient has a coronavirus infection.

11. The method of claim 1, wherein the patient has a SARS-CoV-2 coronavirus infection (COVID-19).

12. The method of claim 1, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug reduces mortality or increases survival in a population of patients having acute kidney injury.

13. The method of claim 1, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is administered once acute kidney injury is detected in the patient, when serum creatinine of the patient increases by 0.3 mg/dl (26.5 μmol/1) or more in 48 hours, when serum creatinine of the patient rises to at least 1.5-fold from baseline within 7 days; when urine output is less than 0.5 ml/kg/hr for more than 6 hours (e.g., KDIGO criteria), or when any other approved diagnostic for AKI is positive.

14. The method of claim 1, wherein the patient has elevated serum creatinine levels, and the 5-HT₃-targeting drug, 5-HT_{3A}-targeting drug, or the pharmaceutically-acceptable salt or solvate of a 5-HT₃-targeting drug or a 5-HT_{3A}-targeting drug is administered to the patient until serum creatinine levels are normalized in the patient.

15. The method of claim 1, wherein the 5-HT₃-targeting drug, the 5-HT_{3A}-targeting drug, or the pharmaceutically-acceptable salt or solvate of a 5-HT₃-targeting drug or a 5-HT_{3A}-targeting drug is administered by intravenous injection or infusion.

16. The method of claim 1, wherein a total of from 0.5 mg to 200 mg of the 5-HT₃-targeting drug, the 5-HT_{3A}-targeting drug, or the pharmaceutically-acceptable salt or solvate of a 5-HT₃-targeting drug or a 5-HT_{3A}-targeting drug is administered daily to the patient, such as in one or more doses or as a continuous or intermittent infusion.

17. The method of claim 16, wherein a total of from 6 mg to 96 mg of ondansetron, or a pharmaceutically-acceptable salt or solvate thereof is administered daily to the patient.

18. The method of claim 17, wherein from 4 mg to 32 mg of ondansetron, or a pharmaceutically-acceptable salt or solvate thereof is administered every 8 hours to the patient.

19. The method of claim 1, further comprising administering to the patient one or more drugs selected from the group consisting of: warfarin lisinopril; heparin; magnesium sulfate; haloperidol; glucagon; metoprolol; furosemide; and hydralazine, or a pharmaceutically-acceptable salt or solvate of any of the preceding.
